(19) United States
(12) Patent Application Publication
(10) Pub. No.: US 2025/0154317 A1
(43) Pub. Date: May 15, 2025

(54) BRANCHED POLY(AMINE-CO-ESTER) POLYMERS FOR MORE EFFICIENT NUCLEIC EXPRESSION

(71) Applicant: Yale University, New Haven, CT (US)

(72) Inventors: Kwangsoo Shin, New Haven, CT (US); Hee Won Suh, New Haven, CT (US); W. Mark Saltzman, New Haven, CT (US)

(21) Appl. No.: 18/838,932

(22) PCT Filed: Feb. 17, 2023
(86) PCT No.: PCT/US2023/062833

§ 371 Date: Aug. 15, 2024

Related U.S. Application Data
(60) Provisional application No. 63/311,541, filed on Feb. 18, 2022.

(51) Int. Cl. C08G 63/685; A61K 9/51; A61K 48/00; C08G 63/91

(52) U.S. Cl. CPC C08G 63/6856; A61K 9/5123; A61K 48/0041; C08G 63/916

(57) ABSTRACT

Methods to produce branching poly(amine-co-ester) (bPACE) polymers having more than two end groups per single polymer, where the amine content can be controlled by end-group modification, have been developed. The resulting bPACE polymers are particularly well suited for delivery of nucleic acid molecules, showing strong complexation, stability, better transfection and expanded routes of administration. Advantages of bPACE include polyplexes with branched polymer present positive charges, even with large amount of nucleic acid (strong and rigid complexation to limited number of nucleic acid), even in neutral pH (7.4, PBS). Transfection by bPACE performs better than linear PACE at a lower weight ratio, showing high expression in vivo. Advantages of bPACE also include polyplexes with branched polymer present better stability in neutral pH, physiological condition, maintaining transfection abilities.

### CROSS-REFERENCE TO RELATED APPLICATIONS

This application claims benefit of U.S. Provisional Application No. 63/311,541 entitled “BRANCHED POLY(AMINE-CO-ESTER) POLYMERS FOR MORE EFFICIENT NUCLEIC EXPRESSION” filed Feb. 18, 2022 by Yale University, listing inventors Kwangsoo Shin, Hee Won Suh, and W. Mark Saltzman.

### STATEMENT REGARDING FEDERALLY SPONSORED RESEARCH

This invention was made with government support under HL147352 and EB000487 awarded by National Institutes of Health. The government has certain rights in the invention.

### FIELD OF THE INVENTION

The field of the invention is generally related to polymer compositions, particularly particles containing branched polymers, for efficient delivery of diagnostic, prophylactic, and/or therapeutic agents, especially nucleic acids such as mRNA.

### BACKGROUND OF THE INVENTION

Non-viral vectors for gene delivery have attracted much attention in the past several decades due to their potential for limited immunogenicity, ability to accommodate and deliver large size genetic materials, and potential for modification of their surface structures. Major categories of non-viral vectors include cationic lipids and cationic polymers. Cationic lipid-derived vectors, which were pioneered by Felgner and colleagues, represent some of the most extensively investigated systems for non-viral gene delivery (Felgner, et al. PNAS, 84, 7413-7417 (1987)) (Templeton, et al. Nat. Biotechnol. 15, 647-652 (1997)) (Chen, et al. J. Invest. Dermatol. 130, 2790-2798 (2010)).

Cationic polymer non-viral vectors have gained increasing attention because of flexibility in their synthesis and structural modifications for specific biomedical applications. Both cationic lipid and cationic polymer systems deliver genes by forming condensed complexes with negatively charged DNA through electrostatic interactions: complex formation protects DNA from degradation and facilitates its cellular uptake and intracellular traffic into the nucleus.

Polyplexes formed between cationic polymers and DNA are generally more stable than lipoplexes formed between cationic lipids and DNA, but both are often unstable in physiological fluids, which contain serum components and salts, and tend to cause the complexes to break apart or aggregate (Al-Dosari, et al. AAPS J. 11, 671-681 (2009)) (Tros de Ilarduya, et al. Eur. J. Pharm. Sci. 40, 159-170 (2010)). Additionally, although some work indicates that anionic polymers or even naked DNA can provide some level of transfection under certain conditions, transfection by both lipids and polymers usually requires materials with excess charge, resulting in polyplexes or lipoplexes with net positive charges on the surface (Nicol, et al. Gene. Ther. 9, 1351-1358 (2002)) (Schlegel, et al. J. Contr. Rel. 152, 393-401 (2011)) (Liu, et al, AAPS J. 9, E92-E104 (2007)) (Liu, et al. Gene Ther. 6, 1258-1266 (1999)). When injected into the circulatory system in vivo, the positive surface charge initiates rapid formation of complex aggregates with negatively charged serum molecules or membranes of cellular components, which are then cleared by the reticuloendothelial system (RES).

More importantly, many cationic vectors developed so far exhibit substantial toxicity, which has limited their clinical applicability (Tros de Ilarduya, et al. Eur. J. Pharm. Sci. 40, 159-170 (2010)) (Gao, et al. Biomaterials 32, 8613-8625 (2011)) (Felgner, et al. J. Biol. Chem. 269, 2550-2561 (1994)) (Kafil, et al. BioImpacts 1, 23-30 (2011)) (Lv, et al. J Contr. Rel. 114, 100-109 (2006)). This too appears to depend on charge: excess positive charges on the surface of the complexes can interact with cellular components, such as cell membranes, and inhibit normal cellular processes, such as clathrin-mediated endocytosis, activity of ion channels, membrane receptors, and enzymes or cell survival signaling (Gao, et al. Biomaterials 32, 8613-8625 (2011)) (Felgner, et al. Enhanced gene delivery and mechanism studies with a novel series of cationic lipid formulations. J. Biol. Chem. 269, 2550-2561 (1994)) (Kafil, et al. Cytotoxic Impacts of Linear and Branched Polyethylenimine Nanostructures in A431 Cells. BioImpacts 1, 23-30 (2011)).

As a result, cationic lipids often cause acute inflammatory responses in animals and humans, whereas cationic polymers, such as PEI, destabilize the plasma-membrane of red blood cells and induce cell necrosis, apoptosis, and autophagy (Tros de Ilarduya, et al. Eur. J. Pharm. Sci. 40, 159-170 (2010)) (Gao, et al. Biomaterials 32, 8613-8625 (2011)) (Lv, et al. J. Contr. Rel. 114, 100-109 (2006)). Because of these undesirable effects, there is a need for highly efficient non-viral vectors that have lower charge densities.

Synthesis of a family of biodegradable poly(amine-co-esters) formed via enzymatic copolymerization of diesters with amino-substituted diols is discussed in Liu, et al. J. Biomed. Mater. Res. A 96A, 456-465 (2011) and Jiang, Z. Biomacromolecules 11, 1089-1093 (2010). Diesters with various chain length (e.g., from succinate to dodecanedioate) were copolymerized with diethanolamines with either an alkyl (methyl, ethyl, n-butyl, t-butyl) or an aryl (phenyl) substituent on the nitrogen. The high tolerance of the lipase catalyst allowed the copolymerization reactions to complete in one step without protection and deprotection of the amino functional groups. Upon protonation at slightly acidic conditions, these poly(amine-co-esters) readily condense DNA and form nano-sized polyplexes. Screening studies revealed that one of these materials, poly(N-methyldiethyleneamine sebacate) (PMSC), transfected a variety of cells including HEK293, U87-MG, and 9L in vitro, with efficiency comparable to that of leading commercial products, such as Lipofectamine 2000 and PEI14. PMSC had been previously used for gene delivery, but the delivery efficiency of the enzymatically synthesized materials was approximately five orders of magnitude higher than any previously reported (Wang, et al. Biomacromolecules 8, 1028-1037 (2007)) (Wang, et al. Biomaterials 28, 5358-5368 (2007)). However, these poly(amine-co-esters) were not effective for systemic delivery of nucleic acids in vivo. Further, there remains a need to develop non-viral vectors for gene delivery with lower carrier material to gene ratio. To address these limitations, a safe and effective nucleic acid delivery platform is needed.

Therefore, it is an object of the invention to provide improved sustained release in vivo delivery systems for nucleic acids such as mRNA.

### SUMMARY OF THE INVENTION

Methods to produce branching poly(amine-co-ester) (bPACE) polymers having more than two end groups per single polymer, where the amine content can be controlled by end-group modification, have been developed. The resulting bPACE polymers are particularly well suited for delivery of nucleic acid molecules, showing strong complexation, stability, better transfection and expanded routes of administration. Advantages of bPACE include forming polyplexes with branched polymer present positive charges, even with large amount of nucleic acid (strong and rigid complexation to limited number of nucleic acid), even in neutral pH (7.4, PBS). Transfection by bPACE performed better than linear PACE at a lower weight ratio, showing high expression in vivo.

Examples demonstrate synthesis and modification of bPACE polymers, complexation of nucleic acids, and expression of nucleic acids delivered with bPACE both in vitro and in vivo.

### BRIEF DESCRIPTION OF THE DRAWINGS

FIG. 1 is a schematic of lipase-catalyzed synthesis of polyesters and ring opening polymerization.

FIGS. 2A-2D are schematics of branching monomers having two hydroxyl or carboxylic groups, including glycerol (FIG. 2A); citric acid (FIG. 2B); N,N,N′,N′-Tetrakis(2-hydroxyethyl)ethylenediamine (THEED) (FIG. 2C); and triethanolamine (FIG. 2D).

FIG. 3 is a schematic of the synthesis of branched PACE and polyplexes from linear-PACE and branched-PACE, based on degree of branching, pentadecanolide (PDL) contents, and end group modification, and showing the formation of PACE/mRNA and bPACE/mRNA polyplexes.

FIG. 4 is a schematic of end group modification of bPACE, showing activation of carboxylate and hydroxyl group with carbonyldiimidazole (CDI) and conjugate amines to CDI-activated bPACE.

FIGS. 5A, 5B, and 5C are graphs showing Mₙ, M_{w}, (kDa) and PDI of the GPC analysis of bPACE, acid end bPACE (bPACE01, bPACE02, bPACE03, bPACE11, bPACE12, bPACE13, bPACE21, bPACE22, and bPACE23; FIG. 5A), CDI activated modified bPACE (0% PDL) (bPACE01-C, bPACE02-C, bPACE03-C, bPACE11-C, bPACE12-C, bPACE13-C, bPACE21-C, bPACE22-C, and bPACE23-C; FIG. 5B), and end group modified bPACE with 1,3-diamino-2-propanol (bPACE11-E14, bPACE12-E14, bPACE13-E14 bPACE22-E14, bPACE22-E14, bPACE23-E14; FIG. 5C).

FIGS. 6A and 6B are graphs of the characterization of polyplex prepared with end-group modified bPACE10 group (PDL 10%) for each of P14, bPl1-E14, bP12-E14, and bP13-E14, respectively, showing zeta potential (mV), Z-average diameter (nm), and polydispersity index (PDI) in sodium Acetate buffer pH 5.8 (25 mM) diluted with DW (to final conc. of 0.5 mM) (FIG. 6A), and the stability of polyplex in phosphate buffered saline (157 mM Na⁺ 140 mM Cl⁻, pH 7.4) (PBS), after six hours of incubation (FIG. 6B).

FIGS. 7A and 7B are graphs showing the characterization of polyplexes. FIGS. 7A-7B are graphs showing characteristics of polyplexes prepared with end-group modified bPACE00 group (PDL 0%), for each of P14, bP01-E14, bP02-E14, and bP03-E14, respectively showing zeta potential (mV), Z-average diameter (nm), and polydispersity index (PDI) (FIG. 7A), and the stability of polyplex in phosphate buffered saline (157 mM Na⁺ 140 mM Cl⁻, pH 7.4) (PBS), after six hours of incubation (FIG. 7B).

FIGS. 8A-8D are graphs showing the characteristics of branched PACE and polyplexes including each of PACE and bPACE. PACE-short is also a linear polymer whose molecular weight is shorter. Therefore, the amount of terminal group of polymers and the contents of the terminal groups after the end-group modification is increased. PACE-s can be considered another control group for comparison to branched PACE, showing that the improvement in polyplexes was the result of branching ND not only from the amount of End group modification. FIG. 8A is a graph of monomer contents (0-60 mol %) for each of PACE-short (PACE-s), PACE. bPACE-1, bPACE-2, bPACE-3, bPACE-4 and bPACE-5, respectively, each including DAP (♦), TEOA (▪), MDEA (▴), SBA (•), and PDL (◯), respectively. FIG. 8B is a graph showing Degree of Branching (0-0.8 DB) for each of PACE-s, PACE, bPACE-1, bPACE-2, bPACE-3, bPACE-4 and bPACE-5, respectively. FIGS. 8C and 8D are bar graphs showing size (0-150 nm) (FIG. 8C); and ζ-potential (0-40 mv) (FIG. 8D) for each of PACE, PACE-s, bPACE-1, bPACE-2, bPACE-3, bPACE-4 and bPACE-5, respectively.

FIGS. 9A-9E are graphs showing mRNA-polymer binding characteristics for polyplexes of PACE, PACE-s, bPACE-1, bPACE-2, bPACE-3, bPACE-4 and bPACE-5, respectively. FIG. 9A is a graph of mRNA binding (%) over polymer/mRNA ratio (w/w) for polyplexes formulated with each of PACE (•), PACE-s (▪), bPACE-1 (▴), bPACE-2 (▾), bPACE-3 (♦), bPACE-4 (O) and bPACE-5 (□), respectively. FIGS. 9B-9C are graphs of stability of EGFP/bPACE polyplexes in serum containing media in terms of transfection ability, showing dissociation constant (K_{d})(FIG. 9B), and maximum specific binding (Bₘₐₓ)(FIG. 9C) for each of PACE, PACE-s, bPACE-1, bPACE-2, bPACE-3, bPACE-4 and bPACE-5, respectively. FIGS. 9D-9E are graphs of results from TNS (6-(p-Toluidino)-2-naphthalenesulfonyl chloride) binding assay for evaluating pKa of polyplex, showing fluorescent intensity (%) over pH for polyplexes formulated with each of PACE (•), PACE-s (▪), bPACE-1 (▴), bPACE-2 (▾), bPACE-3 (♦), bPACE-4 (◯) and bPACE-5 (□), respectively (FIG. 9D); and pKa of each of polyplexes formulated with each of PACE, PACE-s, bPACE-1, bPACE-2, bPACE-3, bPACE-4 and bPACE-5, respectively, from the curve fit shown in FIG. 9F (FIG. 9E).

FIGS. 10A-10G are graphs of mRNA release profiles after treating heparin to mRNA-bPACE polyplexes with various heparin/mRNA to mRNA ratio, analyzed by Ribogreen assay, showing Intensity (a.u) over time (0-6 mins) for each of polyplexes formulated with each of PACE (FIG. 10A), PACE-s (FIG. 10B), bPACE-1 (FIG. 10C), bPACE-2 (FIG. 10D), bPACE-3 (FIG. 10E), bPACE-4 (FIG. 10F) and bPACE-5 (FIG. 10G), respectively, showing traces for each of 0, 0.195, 0.391, 0.781, 1.536, 3.125, 6.25, 12.5, 25, 50, 100 and 200 pg/pg heparin/mRNA.

FIGS. 11A-11C are graphs of colloidal stability of PACE, PACE-s, or bPACE-polyplexes and their transfection ability depending on their storing condition over time. FIGS. 11A-11C are graphs showing changes of hydrodynamic size (FIG. 11A), PDI (FIG. 11B), and zeta-potential (FIG. 11C) of polyplexes formulated with each of PACE (9), PACE-s (n), bPACE-1 (A), bPACE-2 (v), bPACE-3 (*), bPACE-4 (O) and bPACE-5 (Q), respectively, stored at room temperature (22° C.).

FIGS. 12A-12H are graphs quantifying in vitro transfection with GFP mRNA using polyplexes. FIG. 12A is a graph showing mean fluorescence (Mean Fl) of GFP-transfected cells treated with different polymers, including P14; bP11-E14; bP12-E14; bP13-E14; bP01-E14; bP02-E14; and bP03-E14 at different polymer:mRNA ratios including 1:5, 1:10, 1:25, 1:50, and 1:100, respectively FIGS. 12B-12C are graphs of quantitated mRNA loading of PACE, PACE-s and bPACEs with Ribogreen assay with mRNA ratio (w/w), showing Mean Fluorescence Intensity (MFI) over incubation time in media (30 min; 1 hour (h); 4 h; 8 h; and 24 h, respectively), for each of Control (Ctrl); and polyplexes of PACE, PACE-s, bPACE-1, bPACE-2, bPACE-3, bPACE-4 and bPACE-5, respectively (FIG. 12B); and EGFP+ (%) over Media incubation time (h) for polyplexes formulated with each of PACE (•), PACE-s (▪), bPACE-1 (▴), bPACE-2 (▾), bPACE-3 (♦), bPACE-4 (◯) and bPACE-5 (□), respectively (FIG. 12C). FIGS. 12D and 12E are graphs of GFP+ (%) (FIG. 12D) or Mean fluorescence intensity (MFI) (FIG. 12E) following transfection of EGFP-mRNA/bPACE polyplexes formulated with 1:40 and 1:25 of mRNA:polymer ratios using control (Ctrl), or polyplexes of each of PACE, PACE-s, bPACE-1, bPACE-2, bPACE-3, bPACE-4 and bPACE-5, respectively. FIGS. 12F-11H are graphs showing Mean fluorescence intensity (MFI) of EGFP signal transfected A549 cells with polyplexes formulated with each of PACE (•), PACE-s (▪), bPACE-1 (▴), bPACE-2 (▾), bPACE-3 (♦), bPACE-4 (◯) and bPACE-5 (□), or LipoMM stored at 4° C. (FIG. 12F), 22° C. (FIG. 12G), and 37° C. (FIG. 12H) over time, respectively, analyzed flow cytometry. (Polyplexes were formulated with 1:25 mRNA to polymer ratio (w/w), n=3, mean±S.D.

FIG. 13 is a graph of level of luminescence (RLU) of tissue via intramuscular (IM) administration of Fluc-loaded polyplexes at a ratio of 1:10 or 1:25, and intratracheal (IT) administration at a ratio of 1:10 and 1:25.

FIG. 14 is a graph of the in vivo delivery by intratracheal administration expression levels of bP01-E14 in the lung (RLU/protein (mg)) at a ratio of 1:10, 1:25, and 1:50.

FIGS. 15A-15B are graphs of FLuc mRNA delivery in vivo using bPACE polyplexes, showing RLU/mg for polyplexes (PACE, PACE-s, bPACE-1, bPACE-2, bPACE-3, bPACE-4 and bPACE-5, respectively) delivered intra-trachealy (IT) directly to the lungs (FIG. 15A); or systemically by IV administration, showing RLU/mg in each of spleen, kidney, lung and liver for polyplexes formed of each of PACE (•), PACE-short (▪), bPACE-1 (▴), bPACE-2 (▾), and bPACE-3 (▪), respectively (FIG. 15B). All Polyplexes were formulated with 1:25 mRNA to polymer ratio (w/w), n=4, mean±S.D.

### DETAILED DESCRIPTION OF THE INVENTION

### I. Definitions

The term particles include microparticles (including microspheres and microcapsules) (dimensions on average 1 micron to less than about 1000 microns) and nanoparticles (nanospheres and nanocapsules) (diameter less than 1 micron). A particle may be spherical or non-spherical and may have a regular or irregular shape. In certain embodiments, populations of the nanoparticles have an average diameter of about 500 nm, 200 nm, 100 nm, 50 nm, or 10 nm. In some embodiments, the average diameter of the particles is from about 200 nm to about 600 nm, preferably from about 200 to about 500 nm. The term “diameter” is used herein to refer to either of the physical diameter or the hydrodynamic diameter. The diameter of an essentially spherical particle may refer to the physical or hydrodynamic diameter. The diameter of a non-spherical particle may refer preferentially to the hydrodynamic diameter. The diameter of a non-spherical particle may refer to the largest linear distance between two points on the surface of the particle. When referring to multiple particles, the diameter of the particles typically refers to the average diameter of the particles. Particle diameter can be measured using a variety of techniques in the art including, but not limited to, dynamic light scattering.

The terms “active agent” or “biologically active agent” are used interchangeably to refer to a chemical or biological compound that induces a desired pharmacological and/or physiological effect, which may be prophylactic, therapeutic, or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind activated macrophages and tumor-associated microglia (TAMs). The term “prophylactic agent” generally refers to an agent that can be administered to prevent disease or to prevent certain conditions, such as a vaccine.

The term “therapeutically effective amount” refers to an amount of the therapeutic agent that, when incorporated into and/or onto the delivery system, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. For example, compositions including one or more inhibitors may inhibit or reduce the activity and/or quantity of the target by about 10%, 20%, 30%, 40%, 50%, 75%, 85%, 90%, 95%, or 99% from the activity and/or quantity of the same cells in equivalent tissues of subjects that did not receive, or were not treated with the compositions. In some embodiments, the inhibition and reduction are compared at levels of mRNAs, proteins, cells, tissues, and organs.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The phrase “sustained release” refers to release of a substance over an extended period of time, in contrast to a bolus type administration in which the majority of the substance is made biologically available at one time.

The phrases “parenteral administration” and “administered parenterally” are art-recognized terms, and include modes of administration other than enteral and topical administration, typically by injection, and can include intravenous, intramuscular, intrapleural, intravascular, intradermal, intraperitoneal, transtracheal, and subcutaneous injection and infusion.

The term “surfactant” as used herein refers to an agent that lowers the surface tension of a liquid.

The term “transient” refers to expression of a non-integrated transgene for a period of hours, days or weeks, wherein the period of time of expression is less than the period of time for expression of the gene if integrated into the genome or contained within a stable plasmid replicon in the host cell.

A “promoter site” refers to a sequence of nucleotides to which an RNA polymerase, such as the DNA-dependent RNA polymerase originally isolated from bacteriophage, described by Davanloo, et al., Proc. Natl. Acad. Sci. USA, 81:2035-39 (1984), or from another source, binds with high specificity, as described by Chamberlin, et al., Nature, 228:227-231 (1970).

A “poly(A)” refers to a series of adenosines attached by polyadenylation to the mRNA. In the preferred embodiment of a construct for transient expression, the polyA is between 50 and 5000, preferably greater than 64, more preferably greater than 100, most preferably greater than 300 or 400. poly(A) sequences can be modified chemically or enzymatically to modulate mRNA functionality such as localization, stability or efficiency of translation.

The term an “open reading frame” or “ORF” is a series of nucleotides that contains a sequence of bases that could potentially encode a polypeptide or protein. An open reading frame is located between the start-code sequence (initiation codon or start codon) and the stop-codon sequence (termination codon).

The term “construct” refers to a recombinant genetic molecule having one or more isolated polynucleotide sequences.

The term “expression control sequence” refers to a nucleic acid sequence that controls and regulates the transcription and/or translation of another nucleic acid sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides contain as amino acids. Amino acid sequences are written left to right in the direction from the amino to the carboxy terminus, and denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides which do not significantly alter the characteristics of the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties. In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Substitution of like amino acids can also be made on the basis of hydrophilicity, particularly where the biological functional equivalent polypeptide or peptide thereby created is intended for use in immunological embodiments. Amino acid substitutions are generally based on the relative similarity of the amino acid side-chain substituents, for example, their hydrophobicity, hydrophilicity, charge, and size.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The terms “lactone” and “lactone unit” are used to describe a chemical compound that includes a cyclic ester, or the open chain chemical structure that results from the cleavage of the ester bond in the cyclic ester. For example, lactone is used to describe the cyclic ester shown below, and the corresponding lactone-derived open chain structure:

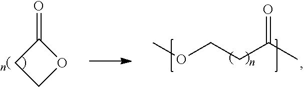

n being an integer. The open chain structure is formed via methods known in the art, including but not limited to, solvolysis, such as hydrolysis, and enzymatic cleavage.

The term “alkyl” refers to the radical of saturated aliphatic groups, including straight-chain alkyl groups and branched-chain alkyl groups. In preferred embodiments, a straight chain or branched chain alkyl has 30 or fewer carbon atoms in its backbone (e.g., C1-C30 for straight chains, C3-C30 for branched chains), preferably 20 or fewer, more preferably 15 or fewer, most preferably 10 or fewer. All integer values of the number of backbone carbon atoms between one and 30 are contemplated and disclosed for the straight chain or branched chain alkyls.

The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

It will be understood by those skilled in the art that the moieties substituted on the hydrocarbon chain can themselves be substituted, if appropriate. For example, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Cycloalkyls can be substituted in the same manner.

“Aryl” refers to C5-C10-membered aromatic, fused aromatic, or biaromatic systems. In some forms, the ring systems have 3-50 carbon atoms. Broadly defined, “aryl”, as used herein, includes 5-, 6-, 7-, 8-, 9-, 10- and 24-membered single-ring aromatic groups that may include from zero to four heteroatoms, for example, benzene, naphthalene, anthracene, phenanthrene, chrysene, pyrene, corannulene, coronene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

“Alkoxy” refers to an alkyl group as defined above with the indicated number of carbon atoms attached through an oxygen bridge. Examples of alkoxy include, but not limited to, methoxy, ethoxy, n-propoxy, i-propoxy, n-butoxy, s-butoxy, n-pentoxy, s-pentoxy, and derivatives thereof.

Primary amines arise when one of three hydrogen atoms in ammonia is replaced by a substituted or unsubstituted alkyl or a substituted or unsubstituted aryl group. Secondary amines have two organic substituents (substituted or unsubstituted alkyl, substituted or unsubstituted aryl or combinations thereof) bound to the nitrogen together with one hydrogen. In tertiary amines, nitrogen has three organic substituents.

“Substituted”, as used herein, means one or more atoms or groups of atoms on the monomer has been replaced with one or more atoms or groups of atoms which are different than the atom or group of atoms being replaced. In some embodiments, the one or more hydrogens on the monomer is replaced with one or more atoms or groups of atoms. Examples of functional groups which can replace hydrogen are listed above in the definition. In some embodiments, one or more functional groups can be added which vary the chemical and/or physical property of the resulting monomer/polymer, such as charge or hydrophilicity/hydrophobicity, etc. Exemplary substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, nitro, heterocyclyl, aralkyl, or an aromatic or heteroaromatic moiety.

### II. Compositions

### A. Branched PACE Polymers

The branched PACE (bPACE) polymers contain a lactone unit composition between about 0% and about 40%, between about 0% and about 30%, or between about 0% and about 20%, such as about 0%, about 5%, about 10%, about 15%, or about 20%. This composition is calculated as lactone unit vs (lactone unit+diester/diacid). Expressed in terms of a molar ratio, the lactone unit vs. (lactone unit+diester/diacid) content is between about 0 and about 0.4, i.e., x/(x+q) is between about 0 and about 0.4, about 0 and about 0.3, about 0 and about 0.2, such as about 0, about 0.05, about 0.1, about 0.15, or about 0.20. The bPACE polymers contain a structure of Formula I:

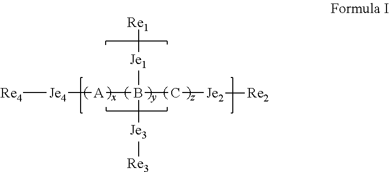

wherein:
Re1, Re2, Re3, and Re4 are independently absent or chemical entities containing a hydroxyl group, a primary amine group, a secondary amine group, a tertiary amine group, or combinations thereof, preferably wherein at least one of Re1, Re2, Re3, and Re4 is present, and preferably wherein the chemical entities have a molecular weight between 10 Da and 2,500 Da, between 10 Da and 1,500 Da, between 10 Da and 1,000 Da, between 10 Da and 500 Da, between 10 and 200 Da, or between 10 and 100 Da,
Je1, Je2, Je3, and Je4 are independently absent, —O—, —S—, —NH—, NRg, —NHC(O)—, —C(O)NH—, —OC(O)—, —C(O)O—, —NRgC(O)—, —C(O)NRg—, —OC(O)NH—, —NHC(O)O—, —OC(O)NRg—, —NRgC(O)O—, —NHC(O)NH—, —NRgC(O)NH—,
   —NHC(O)NRg—, —NRgC(O)NRg—, —SO—, or —S(O)₂—, wherein Rg is, independently for each occurrence, an alkyl, cycloalkyl, heterocycloalkyl, alkylaryl, alkenyl, alkynyl, aryl, or heteroaryl group, optionally substituted with between one and five substituents independently selected from alkyl, cyclopropyl, cyclobutyl ether, amine, halogen, hydroxyl, thiol, ether, thioether, nitrile, CF₃, ester, amide, urea, carbamate, thioether, carboxylic acid, and aryl,
x, y, and z are independently integers from 1 to 1,000, 1 to 500, 1 to 100, 1 to 50, or 1 to 10,
A, B, and C, are, independently for each occurrence, Formula II:

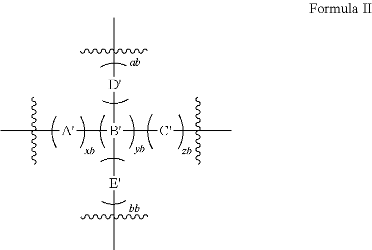

xb and zb are independently integers from 0 to 1,000, with the proviso that xb+zb is at least 1,
yb is independently an integer from 1 to 1,000, ab and bb are independently integers from 0 to 1,000, with the proviso that ab+bb is at least 1, in at least one of A, B, and C,
A′ and C′, independently for each occurrence, contain a structure selected from:

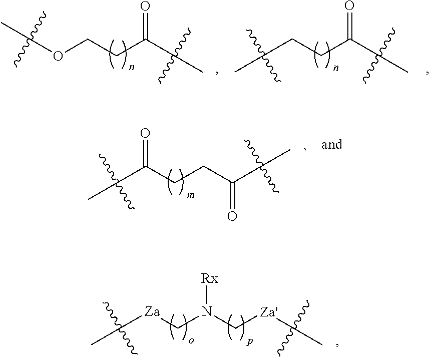

with the proviso that the bPACE contains the structure:
wherein k is 0 or 1,

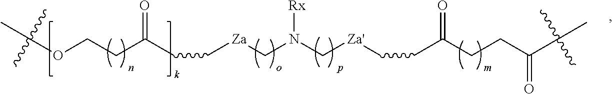

D′ and E′, independently for each occurrence, contain a structure selected from:

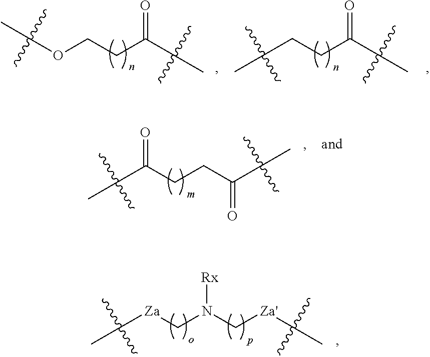

### and B′,

each n is independently an integer from 1-30, m, o, and p are independently integers from 1-20, Za and Za′ are independently O or NR′, wherein Rx is hydrogen, substituted or unsubstituted alkyl, or substituted or unsubstituted aryl, or substituted or unsubstituted alkoxy, wherein R′ is hydrogen, substituted or unsubstituted alkyl, or substituted or unsubstituted aryl,
B′, independently for each occurrence, contains a branch point, preferably containing a structure selected from:

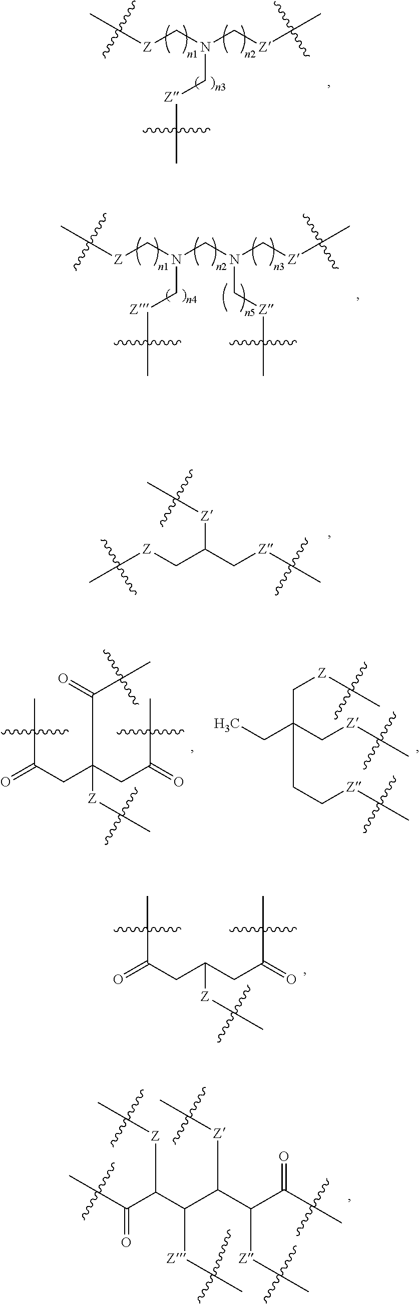

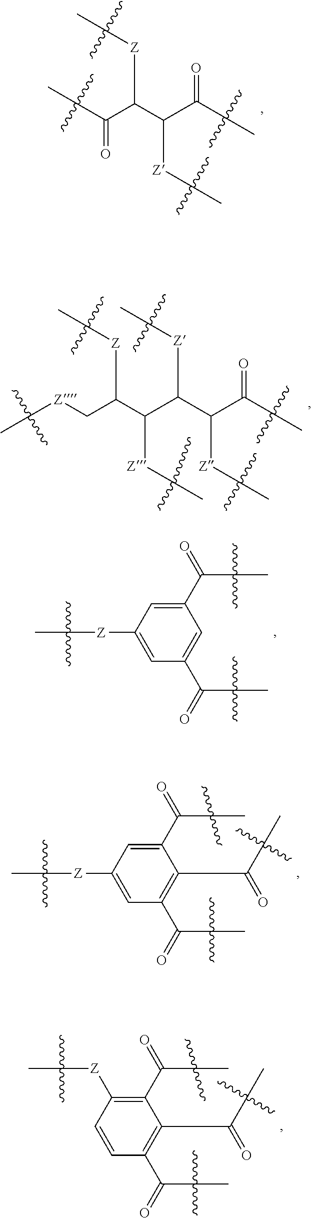

and a combination thereof, wherein each Z, Z′, Z″, Z′″, and Z″″ is independently O, S, or NRy, wherein Ry is hydrogen, substituted or unsubstituted alkyl, substituted or unsubstituted aryl, and each n1, n2, n3, n4, and n5 is independently an integer from 1 to 20, 1 to 15, 1 to 10, or 1 to 5, such as 1, 2, 3, 4, 5, 6, 7, 8, 9, 10, 11, 12, 13, 14, 15, 16, 17, 18, 19, and 20. Preferably, Ry is hydrogen, substituted or unsubstituted alkyl.

In some forms of the bPACE polymers, n is independently an integer from 1 to 24. In some forms of the bPACE polymers, n is independently an integer from 4 to 24, 8 to 22, or 10 to 14, such as 4 (caprolactone), 10 (dodecalactone), 13 (pentadecalactone), or 14 (hexadecalactone).

In some forms of the bPACE polymers, m is an integer from 1 to 10, such as 4, 5, 6, 7, or 8. In some forms of the bPACE polymers, m is 7 (sebacic acid).

In some forms of the bPACE polymers, Rx is hydrogen, substituted or unsubstituted alkyl, or substituted or unsubstituted aryl. In some forms of the bPACE polymers, Za and Za′ are O.

In some forms of the bPACE polymers, o and p are independently integers from 1 to 10, or 1 to 6, such as 1, 2, 3, 4, 5, 6, 7, 8, 9, or 10. In some forms of the bPACE polymers, o and p are the same integer from 1-6, such 2, 3, or 4.

In some forms of the bPACE polymers, n is 13 (e.g., pentadecalactone, PDL), m is 7 (e.g., sebacic acid), Rx is unsubstituted alkyl, Za and Za′ are O, and o and p are 2 (e.g., N-methyldiethanolamine, MDEA).

In some forms of the bPACE polymers, the branched point contains a structure selected from

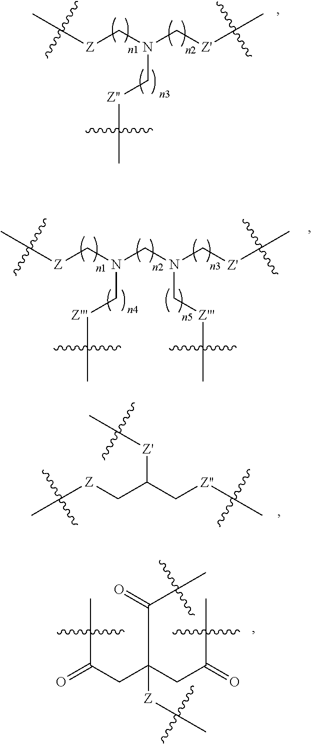

and a combination thereof.

In some forms of the bPACE polymers, for a given branch point, Z, Z′, Z″, Z′″, and Z″″, when present, are independently O, S, or NRy, wherein Ry is hydrogen, substituted or unsubstituted alkyl, substituted or unsubstituted aryl.

In some forms of the bPACE polymers, each n1, n2, n3, n4, and n5 is independently an integer from 1 to 10, such as 1, 2, 3, 4, 5, 6, 7, 8, 9, or 10.

In some forms of the bPACE polymers, for a given branch point, Z, Z′, Z″, Z′″, and Z″″, when present, are the same. In some forms of the bPACE polymers, for a given branch point, Z, Z′, Z″, Z′″, and Z″″, when present, are O.

In some forms of the bPACE polymers the branched point contains a structure selected from:

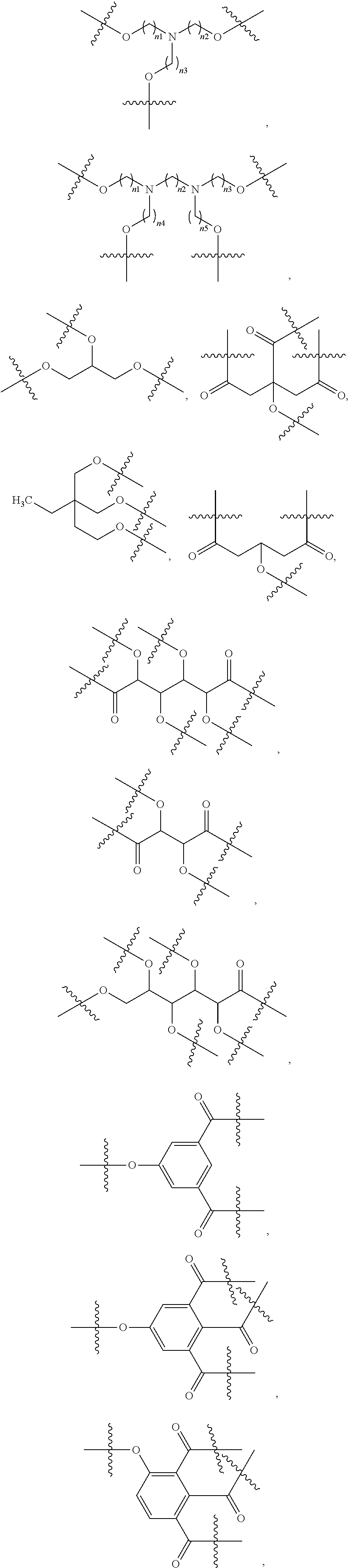

and a combination thereof, wherein n1, n2, n3, n4, and n5 are 2.

In some forms of the bPACE polymers, the branched point contains a structure selected from:

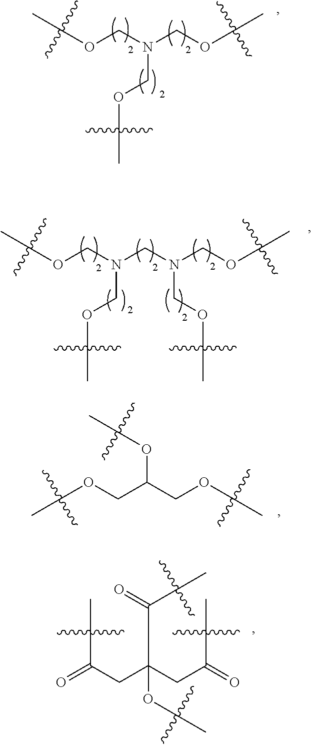

and a combination thereof.

In some forms of the bPACE polymers, Je1, Je2, Je3, and Je4 are independently absent, —O—, —S—, —NH—, NRg, —NHC(O)—, —C(O)NH—, —OC(O)—, —C(O)O—, —NRgC(O)—, or —C(O)NRg—, wherein Rg is, independently for each occurrence, an alkyl, cycloalkyl, heterocycloalkyl, alkylaryl, alkenyl, alkynyl, aryl, or heteroaryl group, optionally substituted with between one and five substituents independently selected from alkyl, cyclopropyl, cyclobutyl ether, amine, halogen, hydroxyl, thiol, ether, thioether, nitrile, CF₃, ester, amide, urea, carbamate, thioether, carboxylic acid, and aryl.

In some forms of the bPACE polymers, Je1, Je2, Je3, and Je4 are independently absent, —O—, —S—, —NH—, —NHC(O)—, —C(O)NH—, —OC(O)—, or —C(O)O—. In some forms of the bPACE polymers, Je1, Je2, Je3, and Je4 are independently —O—, —NH—, —NHC(O)—, —C(O)NH—, —OC(O)—, or —C(O)O—.

In some forms of the bPACE polymers, Re1, Re2, Re3, and Re4 are independently absent, or substituted alkyl containing a hydroxyl group, a primary amine group, a secondary amine group, a tertiary amine group, or combinations thereof. In some forms, the molecular weight of Re1, Re2, Re3, and Re4 are between 10 Da and 500 Da, between 10 Da and 200 Daltons, or between 10 Da and 100 Da.

In some forms of the bPACE polymers, Re1, Re2, Re3, Re4, or a combination thereof contain a primary amine group. In some forms of the bPACE polymers, Re1, Re2, Re3, Re4, or a combination thereof contain a primary amine group and one or more secondary or tertiary amine groups.

In some forms of the bPACE polymers, Re1, Re2, Re3, Re4, or a combination thereof contain a hydroxyl group. In some forms of the bPACE polymers, Re1, Re2, Re3, Re4, or a combination thereof contain a hydroxyl group and one or more amine groups (such as primary, secondary, or tertiary amine groups). In some forms of the bPACE polymers, Re1, Re2, Re3, Re4, or a combination thereof contain a hydroxyl group and no amine group. In some forms of the bPACE polymers, at least one of Re1, Re2, Re3, and Re4 does not contain a hydroxyl group.

In some forms of the bPACE polymers, Re1, Re2, Re3, Re4, or a combination thereof are -unsubstituted C₁-C₁₀ alkylene-Aq-unsubstituted C1-C₁₀ alkylene-Bq, -unsubstituted C₁-C₁₀ alkylene-Aq-substituted C₁-C₁₀ alkylene-Bq, -substituted C₁-C₁₀ alkylene-Aq-unsubstituted C₁-C₁₀ alkylene-Bq, or -substituted C₁-C₁₀ alkylene-Aq-substituted C₁-C₁₀ alkylene-Bq, wherein Aq is absent or —NR_{5b}—, and Bq is hydroxyl, primary amine, secondary amine, or tertiary amine, wherein R_{5b} is hydrogen, substituted alkyl (such as C₁-C₁₀ substituted alkyl), unsubstituted alkyl (such as C₁-C₁₀ unsubstituted alkyl), substituted aryl, or unsubstituted aryl.

In some forms of the bPACE polymers, Re1, Re2, Re3, Re4, or a combination thereof are selected from

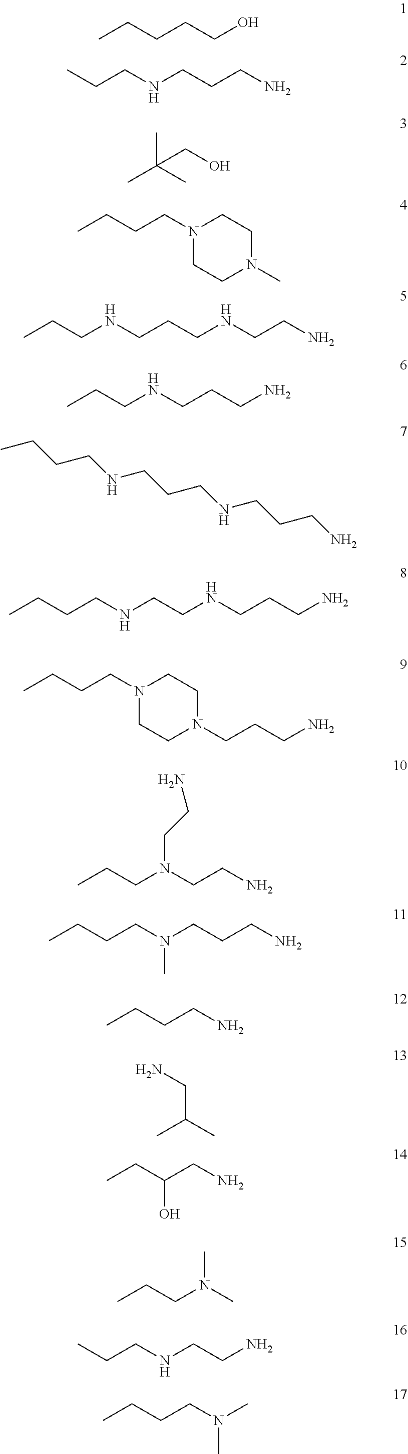

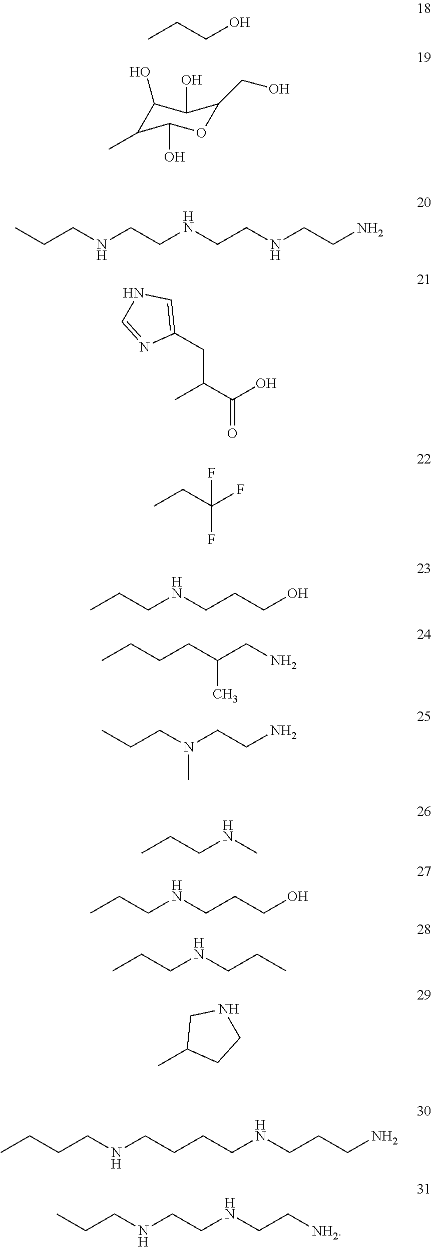

In some forms of the bPACE polymers, Re1, Re2, Re3, Re4, or a combination thereof contains

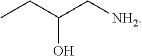

In some forms of the bPACE polymers, where at least two Re1, Re2, Re3, and Re4 are present, the at least two Re1, Re2, Re3, and Re4 are identical.

### B. Therapeutic, Prophylactic and Diagnostic Agents

The bPACE polymers can form various polymer compositions, which are useful for preparing a variety of biodegradable medical devices and for drug delivery. Devices prepared from the polymers can be used for a wide range of different medical applications. Examples of such applications include controlled release of therapeutic, prophylactic, or diagnostic agents; drug delivery; tissue engineering scaffolds; cell encapsulation; targeted delivery; biocompatible coatings; biocompatible implants; guided tissue regeneration; wound dressings; orthopedic devices; prosthetics and bone cements (including adhesives and/or structural fillers); and diagnostics.

The polymers can be used to encapsulate, be mixed with, or be ionically or covalently coupled to any of a variety of therapeutic, prophylactic, or diagnostic agents. A wide variety of biologically active materials can be encapsulated or incorporated, either for delivery to a site by the polymer, or to impart properties to the polymer, such as bioadhesion, cell attachment, enhancement of cell growth, inhibition of bacterial growth, and prevention of clot formation.

In some forms, the agent to be encapsulated and delivered can be a small molecule agent (i.e., non-polymeric agent having a molecular weight less than 2,000, 1500, 1,000, 750, or 500 Dalton) or a macromolecule (e.g., an oligomer or polymer) such as proteins, peptides, nucleic acids, etc. Suitable small molecule active agents include organic, inorganic, and/or organometallic compounds. The polymers can be used for in vivo and/or in vitro delivery of the agent, for example, in a form of particles or micelles.

Examples of suitable therapeutic and prophylactic agents include synthetic inorganic and organic compounds, proteins and peptides, polysaccharides and other sugars, lipids, and DNA and RNA nucleic acid sequences having therapeutic, prophylactic, or diagnostic activities. Nucleic acid sequences include genes, antisense molecules which bind to complementary DNA to inhibit transcription, and ribozymes. Compounds with a wide range of molecular weight can be encapsulated, for example, between 100 and 500,000 grams or more per mole. In the preferred embodiment, the polymers are complexed to nucleic acids.

Exemplary therapeutic agents that can be incorporated into the particles include, but are not limited to tumor antigens, CD4+ T-cell epitopes, cytokines, chemotherapeutic agents, radionuclides, small molecule signal transduction inhibitors, photothermal antennas, monoclonal antibodies, immunologic danger signaling molecules, other immunotherapeutics, enzymes, antibiotics, antivirals (especially protease inhibitors alone or in combination with nucleosides for treatment of HIV or Hepatitis B or C), anti-parasitics (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, immunomodulators (including ligands that bind to Toll-Like Receptors to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the particles into cells (including dendritic cells and other antigen-presenting cells), nutraceuticals such as vitamins, and oligonucleotide drugs (including DNA, RNAs, antisense, aptamers, small interfering RNAs, ribozymes, external guide sequences for ribonuclease P, and triplex forming agents).

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), antibodies to vascular endothelial growth factor (VEGF) such as bevacizumab (AVASTIN®), other anti-VEGF compounds; thalidomide (THALOMID®) and derivatives thereof such as lenalidomide (REVLIMID®); endostatin; angiostatin; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-a or transforming growth factor-P inhibitors, and antibodies to the epidermal growth factor receptor such as panitumumab (VECTIBIX®) and cetuximab (ERBITUX®).

Exemplary immunomodulatory agents include cytokines, xanthines, interleukins, interferons, oligodeoxynucleotides, glucans, growth factors (e.g., TNF, CSF, GM-CSF and G-CSF), hormones such as estrogens (diethylstilbestrol, estradiol), androgens (testosterone, HALOTESTIN® (fluoxymesterone)), progestins (MEGACE® (megestrol acetate), PROVERA® (medroxyprogesterone acetate)), and corticosteroids (prednisone, dexamethasone, hydrocortisone).

Examples of immunological adjuvants that can be associated with the particles include, but are not limited to, TLR ligands, C-Type Lectin Receptor ligands, NOD-Like Receptor ligands, RLR ligands, and RAGE ligands. TLR ligands can include lipopolysaccharide (LPS) and derivatives thereof, as well as lipid A and derivatives there of including, but not limited to, monophosphoryl lipid A (MPL), glycopyranosyl lipid A, PET-lipid A, and 3-O-desacyl-4′-monophosphoryl lipid A.

The particles may also include antigens and/or adjuvants (i.e., molecules enhancing an immune response). Peptide, protein, and DNA based vaccines may be used to induce immunity to various diseases or conditions. Cell-mediated immunity is needed to detect and destroy virus-infected cells. Most traditional vaccines (e.g. protein-based vaccines) can only induce humoral immunity. DNA-based vaccine represents a unique means to vaccinate against a virus or parasite because a DNA based vaccine can induce both humoral and cell-mediated immunity. In addition, DNA based vaccines are potentially safer than traditional vaccines. DNA vaccines are relatively more stable and more cost-effective for manufacturing and storage. DNA vaccines consist of two major components—DNA carriers (or delivery vehicles) and DNAs encoding antigens. DNA carriers protect DNA from degradation, and can facilitate DNA entry to specific tissues or cells and expression at an efficient level.

Representative diagnostic agents are agents detectable by x-ray, fluorescence, magnetic resonance imaging, radioactivity, ultrasound, computer tomography (CT) and positron emission tomography (PET). Ultrasound diagnostic agents are typically a gas such as air, oxygen, or perfluorocarbons. In a preferred embodiment, the polymers are used for delivery of nucleic acids.

### 1. Polynucleotides

As discussed in Examples, the polymers can be used to transfect cells with nucleic acids. The polynucleotide can encode one or more proteins, functional nucleic acids, or combinations thereof. The polynucleotide can be monocistronic or polycistronic. In some embodiments, polynucleotide is multigenic.

In some embodiments, the polynucleotide is transfected into the cell and remains extrachromosomal. In some embodiments, the polynucleotide is introduced into a host cell and is integrated into the host cell's genome. The compositions can be used in methods of gene therapy.

Methods of gene therapy can include the introduction into the cell of a polynucleotide that alters the genotype of the cell. Introduction of the polynucleotide can correct, replace, or otherwise alter the endogenous gene via genetic recombination. Methods can include introduction of an entire replacement copy of a defective gene, a heterologous gene, or a small nucleic acid molecule such as an oligonucleotide. For example, a corrective gene can be introduced into a non-specific location within the host's genome.

In some embodiments, the polynucleotide is incorporated into or part of a vector. Methods to construct expression vectors containing genetic sequences and appropriate transcriptional and translational control elements are well known in the art. These methods include in vitro recombinant DNA techniques, synthetic techniques, and in vivo genetic recombination. Expression vectors generally contain regulatory sequences and necessary elements for the translation and/or transcription of the inserted coding sequence, which can be, for example, the polynucleotide of interest. The coding sequence can be operably linked to a promoter and/or enhancer to help control the expression of the desired gene product. Promoters used in biotechnology are of different types according to the intended type of control of gene expression. They can be generally divided into constitutive promoters, tissue-specific or development-stage-specific promoters, inducible promoters, and synthetic promoters.

For example, in some embodiments, the polynucleotide of interest is operably linked to a promoter or other regulatory elements known in the art. Thus, the polynucleotide can be a vector such as an expression vector. The engineering of polynucleotides for expression in a prokaryotic or eukaryotic system may be performed by techniques generally known to those of skill in recombinant expression. An expression vector typically comprises one of the compositions under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors. It will be appreciated that any of these vectors may be packaged and delivered using the polymers.

Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the compositions. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

In preferred embodiments, the polynucleotide cargo is an RNA, such as an mRNA. The mRNA can encode a polypeptide of interest. In some embodiments, the mRNA has a cap on the 5′ end and/or a 3′ poly(A) tail which can modulate ribosome binding, initiation of translation and stability mRNA in the cell.

### 2. Polypeptide of Interest

The polynucleotide can encode one or more polypeptides of interest. The polypeptide can be any polypeptide. For example, the polypeptide encoded by the polynucleotide can be a polypeptide that provides a therapeutic or prophylactic effect to an organism or that can be used to diagnose a disease or disorder in an organism. For example, for treatment of cancer, autoimmune disorders, parasitic, viral, bacterial, fungal or other infections, the polynucleotide(s) to be expressed may encode a polypeptide that functions as a ligand or receptor for cells of the immune system, or can function to stimulate or inhibit the immune system of an organism. As discussed in the example below, a polynucleotide encoding TNF-related apoptosis-inducing ligand (TRAIL) can be delivered to tumor cells using the polymeric particles in a method of treating cancer.

In some embodiments, the polynucleotide supplements or replaces a polynucleotide that is defective in the organism.

In some embodiments, the polynucleotide includes a selectable marker, for example, a selectable marker that is effective in a eukaryotic cell, such as a drug resistance selection marker. This selectable marker gene can encode a factor necessary for the survival or growth of transformed host cells grown in a selective culture medium. Typical selection genes encode proteins that confer resistance to antibiotics or other toxins, e.g., ampicillin, neomycin, methotrexate, kanamycin, gentamycin, Zeocin, or tetracycline, complement auxotrophic deficiencies, or supply critical nutrients withheld from the media.

In some embodiments, the polynucleotide includes a reporter gene.

Reporter genes are typically genes that are not present or expressed in the host cell. The reporter gene typically encodes a protein which provides for some phenotypic change or enzymatic property. Examples of such genes are provided in Weising et al. Ann. Rev. Genetics, 22, 421 (1988). Preferred reporter genes include glucuronidase (GUS) gene and GFP genes.

### 3. Functional Nucleic Acids

The polynucleotide can be, or can encode a functional nucleic acid. Functional nucleic acids are nucleic acid molecules that have a specific function, such as binding a target molecule or catalyzing a specific reaction. Functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively, the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

Aptamers are molecules that interact with a target molecule, preferably in a specific way. Typically, aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000-fold difference in binding affinities between the target molecule and another molecule that differ at only a single position on the molecule. It is preferred that the aptamer have a K_{d} with the target molecule at least 10, 100, 1000, 10,000, or 100,000-fold lower than the K_{d} with a background binding molecule. It is preferred when doing the comparison for a molecule such as a polypeptide, that the background molecule be a different polypeptide.

Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

### Composition of the Polynucleotides

The polynucleotide can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally occurring nucleotides comprise uracil, thymine, cytosine, adenine and guanine as the heterocyclic bases, and ribose or deoxyribose sugar linked by phosphodiester bonds.

The polynucleotide can be composed of nucleotide analogs that have been chemically modified to improve stability, half-life, or specificity or affinity for a target sequence, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge. Modifications should not prevent, and preferably enhance, the ability of the oligonucleotides to enter a cell and carry out a function such inhibition of gene expression as discussed above.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

As discussed in more detail below, in a preferred embodiment, the oligonucleotide is a morpholino oligonucleotide.

a. Heterocyclic Bases

The principal naturally-occurring nucleotides include uracil, thymine, cytosine, adenine and guanine as the heterocyclic bases. The oligonucleotides can include chemical modifications to their nucleobase constituents. Chemical modifications of heterocyclic bases or heterocyclic base analogs may be effective to increase the binding affinity or stability in binding a target sequence. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-beta-D-ribofuranosyl)pyridine (2-aminopyridine), and various pyrrolo- and pyrazolopyrimidine derivatives.

b. Sugar Modifications

Polynucleotides can also contain nucleotides with modified sugar moieties or sugar moiety analogs. Sugar moiety modifications include, but are not limited to, 2′-O-aminoetoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O-(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

The polynucleotide can be a morpholino oligonucleotide. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil, or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tₘ, even with oligomers as short as 10-14 bases; the ability of the oligomer to be actively transported into mammalian cells; and the ability of an oligomer:RNA heteroduplex to resist RNAse degradation. In some embodiments, oligonucleotides employ morpholino-based subunits bearing base-pairing moieties, joined by uncharged linkages.

c. Internucleotide Linkages

Internucleotide bond refers to a chemical linkage between two nucleoside moieties. Modifications to the phosphate backbone of DNA or RNA oligonucleotides may increase the binding affinity or stability polynucleotides, or reduce the susceptibility of polynucleotides to nuclease digestion. Cationic modifications, including, but not limited to, diethyl-ethylenediamide (DEED) or dimethyl-aminopropylamine (DMAP) may be especially useful due to decrease electrostatic repulsion between the oligonucleotide and a target. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

Examples of modified nucleotides with reduced charge include modified internucleotide linkages such as phosphate analogs having achiral and uncharged intersubunit linkages (e.g., Sterchak, et al., Organic Chem., 52:4202, (1987)), and uncharged morpholino-based polymers having achiral intersubunit linkages (see, e.g., U.S. Pat. No. 5,034,506), as discussed above. Some internucleotide linkage analogs include morpholidate, acetal, and polyamide-linked heterocycles.

In another embodiment, the oligonucleotides are composed of locked nucleic acids. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

In some embodiments, the oligonucleotides are composed of peptide nucleic acids. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications include peptide and amino acid variations and modifications. Thus, the backbone constituents of oligonucleotides such as PNA may be peptide linkages, or alternatively, they may be non-peptide peptide linkages. Examples include acetyl caps, amino spacers such as 8-amino-3,6-dioxaoctanoic acid (referred to herein as 0-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571, and 5,786,571.

Polynucleotides optionally include one or more terminal residues or modifications at either or both termini to increase stability, and/or affinity of the oligonucleotide for its target. Commonly used positively charged moieties include the amino acids lysine and arginine, although other positively charged moieties may also be useful. For example, lysine and arginine residues can be added to a bis-PNA linker or can be added to the carboxy or the N-terminus of a PNA strand. Polynucleotides may further be modified to be end capped to prevent degradation using a 3′ propylamine group. Procedures for 3′ or 5′ capping oligonucleotides are well known in the art.

### III. Formulations

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

For detailed information concerning materials, equipment, and processes for preparing tablets and delayed release dosage forms, see Pharmaceutical Dosage Forms: Tablets, eds. Lieberman et al., (New York: Marcel Dekker, Inc., 1989), and Ansel et al., Pharmaceutical Dosage Forms and Drug Delivery Systems, 6.sup.th Ed. (Media, PA: Williams & Wilkins, 1995).

### IV. Methods of Preparing Particles

Particles can be prepared using a variety of techniques known in the art. The technique to be used can depend on a variety of factors including the polymer used to form the nanoparticles, the desired size range of the resulting particles, and suitability for the material to be encapsulated.

Methods known in the art that can be used to prepare nanoparticles include, but are not limited to, polyelectrolyte condensation (see Suk, et al., Biomaterials, 27, 5143-5150 (2006)); single and double emulsion; nanoparticle molding, and electrostatic self-assembly (e.g., polyethylene imine-DNA or liposomes). Preferred methods also include microfluidic methods (see, for example, Li, et al., Nano Micro Small Review (2019); Anal. Chem. 2018, 90, 3, 1434-1443 (2017); Lim, et al., “Ultra-High Throughput Synthesis of Nanoparticles with Homogeneous Size Distribution Using a Coaxial Turbulent Jet Mixer” ACS Nano 2014, 8, 6, 6056-6065 (2014)). These also work for PACE nanoparticles.

In one embodiment, the loaded particles are prepared by combining a solution of the polymer, typically in an organic solvent, with the polynucleotide of interest. The polymer solution is prepared by dissolving or suspending the polymer in a solvent. The solvent should be selected so that it does not adversely affect (e.g., destabilize or degrade) the nucleic acid to be encapsulated and which are present in the final product at a level deemed acceptable by regulatory agencies such as the Food and Drug Administration. Suitable solvents include, but are not limited to, DMSO and methylene chloride. The concentration of the polymer in the solvent can be varied as needed. In some embodiments, the concentration is for example 25 mg/ml. The polymer solution can also be diluted in a buffer, for example, sodium acetate buffer.

Next, the polymer solution is mixed with the agent to be encapsulated, such as a polynucleotide. The agent can be dissolved in a solvent to form a solution before combining it with the polymer solution. In some embodiments, the agent is dissolved in a physiological buffer before combining it with the polymer solution. The ratio of polymer solution volume to agent solution volume can be 1:1. The combination of polymer and agent are typically incubated for a few minutes to form particles before using the solution for its desired purpose, such as transfection. For example, a polymer/polynucleotide solution can be incubated for 2, 5, 10, or more than 10 minutes before using the solution for transfection. The incubation can be at room temperature.

In some embodiments, the particles are also incubated with a solution containing a coating agent prior to use. The particle solution can be incubated with the coating agent for 2, 5, 10, or more than 10 minutes before using the particles for transfection. The incubation can be at room temperature.

In some embodiments, if the agent is a polynucleotide, the polynucleotide is first complexed to a polycation before mixing with polymer. Complexation can be achieved by mixing the polynucleotides and polycations at an appropriate molar ratio. When a polyamine is used as the polycation species, it is useful to determine the molar ratio of the polyamine nitrogen to the polynucleotide phosphate (N/P ratio). In a preferred embodiment, inhibitory RNAs and polyamines are mixed together to form a complex at an N/P ratio of between approximately 1:1 to 1:25, preferably between about 8:1 to 15:1. The volume of polyamine solution required to achieve particular molar ratios can be determined according to the following formula:

$${V}_{NH 2} = \underline{{C}_{inhRNA , final} \times {M}_{w , inhRNA} / {C}_{inhRNA , final} \times {M}_{w , P} \times {Φ}_{N : P} \times Φ {V}_{final}} {C}_{NH 2} / {M}_{w , NH 2}$$

where M_{w,inhRNA}=molecular weight of inhibitory RNA, M_{w,P}=molecular weight of phosphate groups of inhibitory RNA, Φ_{N:P}=N:P ratio (molar ratio of nitrogens from polyamine to the ratio of phosphates from the inhibitory RNA), C_{NH2}, stock=concentration of polyamine stock solution, and M_{w,NH2}=molecular weight per nitrogen of polyamine.

Methods of mixing polynucleotides with polycations to condense the polynucleotide are known in the art. See for example U.S. Published Application No. 2011/0008451.

The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values. Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quartemized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In some embodiments, the polycation is a polyamine. Polyamines are compounds having two or more primary amine groups. Suitable naturally occurring polyamines include, but are not limited to, spermine, spermidine, cadaverine and putrescine. In a preferred embodiment, the polyamine is spermidine.

In another embodiment, the polycation is a cyclic polyamine. Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane) which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

### V. Methods of Using the Particles/Micelles

### A. Delivery of Active Agent

The particles described above can be use for convention polymeric drug delivery, using standard methodology and reagents.

The polymers, or micellular particles or solid polymeric nanoparticles formed thereof can be used to deliver an effective amount of one or more therapeutic, diagnostic, and/or prophylactic agents to a patient in need of such treatment. The amount of agent to be administered can be readily determine by the prescribing physician and is dependent on the age and weight of the patient and the disease or disorder to be treated.

The micellular particles or solid polymeric nanoparticles are useful in drug delivery (exemplary drugs include therapeutic, nutritional, diagnostic, and prophylactic agents), whether injected intravenously, subcutaneously, or intramuscularly, administered to the nasal or pulmonary system, injected into a tumor milieu, administered to a mucosal surface (nasal, pulmonary, vaginal, rectal, buccal, sublingual), or encapsulated for oral delivery. The particles may be administered as a dry powder, as an aqueous suspension (in water, saline, buffered saline, etc), in a hydrogel, organogel, or liposome, in capsules, tablets, troches, or other standard pharmaceutical excipient.

### B. Transfection

The compositions can be for cell transfection of polynucleotides. The transfection can occur in vitro or in vivo, and can be applied in applications including gene therapy and disease treatment. The compositions can be more efficient, less toxic, or a combination thereof when compared to a control. In some embodiments, the control is cells treated with an alternative transfection reagent such as LIPOFECTAMINE 2000 or polyethylenimine (PEI).

Transfection is carried out by contacting cells with the solution containing the polymers associated or complexed with polynucleotides. For in vivo methods, the contacting typically occurs in vivo after the solution is administered to the subject. For in vitro methods, the solution is typically added to a culture of cells and allowed to contact the cells for minutes, hours, or days. The cells can subsequently be washed to move excess polymers or particles thereof.

The polynucleotide delivered by the polymers can be selected by one of skill in the art depending on the condition or disease to be treated. The polynucleotide can be, for example, a gene or cDNA of interest, mRNA, a functional nucleic acid such as an inhibitory RNA, a tRNA, an rRNA, or an expression vector encoding a gene or cDNA of interest, a functional nucleic acid a tRNA, or an rRNA. In some embodiments, two or more polynucleotides are administered in combination. In some forms, the weight ratio of the polymer (e.g., bPACE polymer) to nucleic acid (e.g., a gene or cDNA of interest, mRNA, a functional nucleic acid such as an inhibitory RNA, a tRNA, an rRNA, or an expression vector encoding a gene or cDNA of interest, a functional nucleic acid a tRNA, or an rRNA) is from 100:1 to 5:1.

The compositions can be used in a method of delivering polynucleotides to cells in vitro. For example, the compositions can be used for in vitro transfection of cells. The method typically involves contacting the cells with polymeric particles including a polynucleotide in an effective amount to introduce the polynucleotide into the cell's cytoplasm. In some embodiments, the polynucleotide is delivered to the cell in an effective amount to change the genotype or a phenotype of the cell. The cells can primary cells isolated from a subject, or cells of an established cell line. The cells can be of a homogenous cell type, or can be a heterogeneous mixture of different cell types. For example, the polymeric particles can be introduced into the cytoplasm of cells from a heterogenous cell line possessing cells of different types, such as in a feeder cell culture, or a mixed culture in various states of differentiation. The cells can be a transformed cell line that can be maintained indefinitely in cell culture. Exemplary cell lines are those available from American Type Culture Collection including tumor cell lines.

Any eukaryotic cell can be transfected to produce cells that express a specific nucleic acid, for example a metabolic gene, including primary cells as well as established cell lines. Suitable types of cells include but are not limited to undifferentiated or partially differentiated cells including stem cells, totipotent cells, pluripotent cells, embryonic stem cells, inner mass cells, adult stem cells, bone marrow cells, cells from umbilical cord blood, and cells derived from ectoderm, mesoderm, or endoderm. Suitable differentiated cells include somatic cells, neuronal cells, skeletal muscle, smooth muscle, pancreatic cells, liver cells, and cardiac cells. In another embodiment, siRNA, antisense polynucleotides (including siRNA or antisense polynucleotides) or inhibitory RNA can be transfected into a cell using the compositions.

The methods are particularly useful in the field of personalized therapy, for example, to repair a defective gene, de-differentiate cells, or reprogram cells. For example, target cells are first isolated from a donor using methods known in the art, contacted with the polymeric particles including a polynucleotide causing a change to the in vitro (ex vivo), and administered to a patient in need thereof. Sources or cells include cells harvested directly from the patient or an allographic donor. In preferred embodiments, the target cells to be administered to a subject will be autologous, e.g., derived from the subject, or syngeneic.

Allogeneic cells can also be isolated from antigenically matched, genetically unrelated donors (identified through a national registry), or by using target cells obtained or derived from a genetically related sibling or parent.

Cells can be selected by positive and/or negative selection techniques. For example, antibodies binding a particular cell surface protein may be conjugated to magnetic beads and immunogenic procedures utilized to recover the desired cell type. It may be desirable to enrich the target cells prior to transient transfection. An enriched population of target cells means a proportion of a desirable element (e.g., the target cell) is higher than that found in the natural source of the cells. A composition of cells may be enriched over a natural source of the cells by at least one order of magnitude, preferably two or three orders, and more preferably 10, 100, 200, or 1000 orders of magnitude. Once target cells have been isolated, they may be propagated by growing in suitable medium according to established methods known in the art. Established cell lines may also be useful in for the methods. The cells can be stored frozen before transfection, if necessary.

Next the cells are contacted with the composition in vitro to repair, de-differentiate, re-differentiate, and/or re-program the cell. The cells can be monitored, and the desired cell type can be selected for therapeutic administration.

Following repair, de-differentiation, and/or re-differentiation and/or reprogramming, the cells are administered to a patient in need thereof. In the most preferred embodiments, the cells are isolated from and administered back to the same patient. In alternative embodiments, the cells are isolated from one patient, and administered to a second patient. The method can also be used to produce frozen stocks of altered cells which can be stored long-term, for later use. In one embodiment, fibroblasts, keratinocytes, or hematopoietic stem cells are isolated from a patient and repaired, de-differentiated, or reprogrammed in vitro to provide therapeutic cells for the patient.

### C. In Vivo Methods

The compositions can be used in a method of delivering polynucleotides to cells in vivo. It has been discovered that the bPACE polymers are more efficient and/or less toxic for systemic in vivo transfection of polynucleotides than alternative transfection reagents include LIPOFECTAMINE 2000, PEI, and even other PMSCs. Accordingly, in some embodiments, the cell specific polymeric particles including a therapeutic polynucleotide are administered systemically in vivo to a treat a disease, for example cancer.

In some in vivo approaches, the compositions are administered to a subject in a therapeutically effective amount. The precise dosage will vary according to a variety of factors such as subject-dependent variables (e.g., age, immune system health, etc.), the disease, and the treatment being affected.

Pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, pulmonary, or sublingual) routes of administration or using bioerodible inserts and can be formulated in dosage forms appropriate for each route of administration.

In some embodiments, the compositions are administered systemically, for example, by injection or pulmonary administration, in an amount effective for delivery of the compositions to targeted cells. Other routes include trans-dermal or oral.

In certain embodiments, the compositions are administered locally, for example by injection directly into a site to be treated. Typically, local injection causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the polymeric particles to the immediate area of the implant.

The polymeric particles can be provided to the cell either directly, such as by contacting it with the cell, or indirectly, such as through the action of any biological process. For example, the polymeric particles can be formulated in a physiologically acceptable carrier or vehicle, and injected into a tissue or fluid surrounding the cell. The polymeric particles can cross the cell membrane by simple diffusion, endocytosis, or by any active or passive transport mechanism.

The compositions can be used in gene therapy protocols for the treatment of gene related diseases or disorders. Cell dysfunction can also be treated or reduced using the compositions and methods. In some embodiments, diseases amenable to gene therapy are specifically targeted. The disease can be in children, for example individuals less than 18 years of age, typically less than 12 years of age, or adults, for example individuals 18 years of age or more. In some embodiments, methods are used to treat a host diagnosed with a disease, by transfection of the polymeric particles including a polynucleotide into the cell affected by the disease and wherein the polynucleotide encodes a therapeutic protein. In another embodiment, an inhibitory RNA is directed to a specific cell type or state to reduce or eliminate the expression of a protein, thereby achieving a therapeutic effect. The methods encompass manipulating, augmenting, or replacing genes to treat diseases caused by genetic defects or abnormalities, as well as to make vaccines for treating infectious disease or cancers.

Suitable genetic based diseases that can be treated with the compositions herein include but are not limited to:

### Mitochondrial Disease:

Alpers Disease; Barth syndrome; P-oxidation defects; carnitine-acyl-carnitine deficiency; carnitine deficiency; co-enzyme Q10 deficiency; Complex I deficiency; Complex II deficiency; Complex III deficiency; Complex IV deficiency; Complex V deficiency; cytochrome c oxidase (COX) deficiency, LHON—Leber Hereditary Optic Neuropathy; MM—Mitochondrial Myopathy; LIMM—Lethal Infantile Mitochondrial Myopathy; MMC—Maternal Myopathy and Cardiomyopathy; NARP—Neurogenic muscle weakness, Ataxia, and Retinitis Pigmentosa; Leigh Disease; FICP—Fatal Infantile Cardiomyopathy Plus, a MELAS-associated cardiomyopathy; MELAS—Mitochondrial Encephalomyopathy with Lactic Acidosis and Strokelike episodes; LDYT—Leber's hereditary optic neuropathy and Dystonia; MERRF—Myoclonic Epilepsy and Ragged Red Muscle Fibers; MHCM—Maternally inherited Hypertrophic CardioMyopathy; CPEO—Chronic Progressive External Ophthalmoplegia; KSS—Kearns Sayre Syndrome; DM—Diabetes Mellitus; DMDF Diabetes Mellitus+DeaFness; CIPO—Chronic Intestinal Pseudoobstruction with myopathy and Ophthalmoplegia; DEAF—Maternally inherited DEAFness or aminoglycoside-induced DEAFness; PEM—Progressive encephalopathy; SNHL—SensoriNeural Hearing Loss; Encephalomyopathy; Mitochondrial cytopathy; Dilated Cardiomyopathy; GER—Gastrointestinal Reflux; DEMCHO—Dementia and Chorea; AMDF—Ataxia, Myoclonus; Exercise Intolerance; ESOC Epilepsy, Strokes, Optic atrophy, & Cognitive decline; FBSN Familial Bilateral Striatal Necrosis; FSGS Focal Segmental Glomerulosclerosis; LIMM Lethal Infantile Mitochondrial Myopathy; MDM Myopathy and Diabetes Mellitus; MEPR Myoclonic Epilepsy and Psychomotor Regression; MERME MERRF/MELAS overlap disease; MHCM Maternally Inherited Hypertrophic CardioMyopathy; MICM Maternally Inherited Cardiomyopathy; MILS Maternally Inherited Leigh Syndrome; Mitochondrial Encephalocardiomyopathy; Multisystem Mitochondrial Disorder (myopathy, encephalopathy, blindness, hearing loss, peripheral neuropathy); NAION Nonarteritic Anterior Ischemic Optic Neuropathy; NIDDM Non-Insulin Dependent Diabetes Mellitus; PEM Progressive Encephalopathy; PME Progressive Myoclonus Epilepsy; RTT Rett Syndrome; SIDS Sudden Infant Death Syndrome; MIDD Maternally Inherited Diabetes and Deafness; and MODY Maturity-Onset Diabetes of the Young.

### Nuclear Disease:

Muscular Dystrophies, Ellis-van Creveld syndrome, Marfan syndrome, Myotonic dystrophy, Spinal muscular atrophy, Achondroplasia, Amyotrophic lateral sclerosis, Charcot-Marie-Tooth syndrome, Cockayne syndrome, Diastrophic dysplasia, Duchenne muscular dystrophy, Ellis-van Creveld syndrome, Fibrodysplasia ossificans progressive, Alzheimer disease, Angelman syndrome, Epilepsy, Essential tremor, Fragile X syndrome, Friedreich's ataxia, Huntington disease, Niemann-Pick disease, Parkinson disease, Prader-Willi syndrome, Rett syndrome, Spinocerebellar atrophy, Williams syndrome, Ataxia telangiectasia, Anemia, sickle cell, Burkitt lymphoma, Gaucher disease, Hemophilia, Leukemia, Paroxysmal nocturnal hemoglobinuria, Porphyria, Thalassemia, Crohn's disease, Alpha-1-antitrypsin deficiency, Cystic fibrosis, Deafness, Pendred syndrome, Glaucoma, Gyrate atrophy of the choroid and retina, Adrenal hyperplasia, Adrenoleukodystrophy, Cockayne syndrome, Long QT syndrome, Immunodeficiency with hyper-IgM, Alport syndrome, Ellis-van Creveld syndrome, Fibrodysplasia ossificans progressive, Waardenburg syndrome, Werner syndrome.

### Infectious Disease:

Viral—AIDS, AIDS Related Complex, Chickenpox (Varicella), Common cold, Cytomegalovirus Infection, Colorado tick fever, Dengue fever, Ebola haemorrhagic fever, Epidemic parotitis, Flu, Hand, foot and mouth disease, Hepatitis—Herpes simplex, Herpes zoster, HPV, Influenza, Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease—Yellow fever; Bacterial—Anthrax, Bacterial Meningitis, Brucellosis, Bubonic plague, Campylobacteriosis, Cat Scratch Disease, Cholera, Diphtheria, Epidemic Typhus, Gonorrhea, Hansen's Disease, Legionellosis, Leprosy, Leptospirosis, Listeriosis, Lyme Disease, Melioidosis, MRSA infection, Nocardiosis, Pertussis, Pneumococcal pneumonia, Psittacosis, Q fever, Rocky Mountain Spotted Fever or RMSF, Salmonellosis, Scarlet Fever, Shigellosis, Syphilis, Tetanus, Trachoma, Tuberculosis, Tularemia, Typhoid Fever, Typhus, Whooping Cough; Parasitic—African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, Trypanosomiasis.

### Cancers:

Breast and ovarian cancer, Burkitt lymphoma, Chronic myeloid leukemia, Colon cancer, Lung cancer, Malignant melanoma, Multiple endocrine neoplasia, Neurofibromatosis, p53 LieFrauMeni, Pancreatic cancer, Prostate cancer, retinoblastoma, von Hippel-Lindau syndrome, Polycystic kidney disease, Tuberous sclerosis.

### Metabolic Disorders:

Adrenoleukodystrophy, Atherosclerosis, Best disease, Gaucher disease, Glucose galactose malabsorption, Gyrate atrophy, Juvenile onset diabetes, Obesity, Paroxysmal nocturnal hemoglobinuria, Phenylketonuria, Refsum disease, Tangier disease, Tay-Sachs disease, Adrenoleukodystrophy, Type 2 Diabetes, Gaucher disease, Hereditary hemochromatosis, Lesch-Nyhan syndrome, Maple syrup urine disease, Menkes syndrome, Niemann-Pick disease, Pancreatic cancer, Prader-Willi syndrome, Porphyria, Refsum disease, Tangier disease, Wilson's disease, Zellweger syndrome, progerias, SCID.

### Autoimmune Disorders:

Autoimmune polyglandular syndrome, lupus, type I diabetes, scleroderma, multiple sclerosis, Crohn's disease, chronic active hepatitis, rheumatoid arthritis, Graves' disease, myasthenia gravis, myositis, antiphospholipid syndrome (APS), uveitis, polymyositis, Raynaud's phenomenon, and demyelinating neuropathies, and rare disorders such as polymyalgia rheumatica, temporal arteritis, Sjogren's syndrome, Bechet's disease, Churg-Strauss syndrome, and Takayasu's arteritis.

### Inflammatory Disorders:

Alopecia, Diastrophic dysplasia, Ellis-van Creveld syndrome, Asthma, Arthritis, including osteoarthritis, rheumatoid arthritis, and spondyloarthropathies.

### Age-Related Disorders:

Alzheimer Disease, Parkinson's Disease, Atherosclerosis, Age-Related Macular Degeneration, Age-related Osteoporosis.

The methods and compositions can also be used to treat, manage, or reduce symptoms associated with aging, in tissue regeneration/regenerative medicine, stem cell transplantation, inducing reversible genetic modifications, expressing inhibitory RNA, cognitive enhancement, performance enhancement, and cosmetic alterations to human or non-human animal.

### D. Transgenic Non-Human Animals

The compositions and methods can also be used to generate transgenic non-human animals. In particular, zygote microinjection, nuclear transfer, blastomere electrofusion and blastocyst injection of embryonic stem (ES) cell cybrids have each provided feasible strategies for creating transgenic animals. In one embodiment an embryonic stem (ES) cell is transfected and injected into the blastocyst of a mammalian embryo as a means of generating chimeric mice. In another embodiment, embryonic stem (ES) cell are first prepared, followed by blastocyst injection into embryos. The use of cells carrying specific genes and modifications of interest allows the creation and study of the consequences of the transfected DNA. In theory, this technique offers the prospect of transferring any polynucleotide into a whole organism. For example, the methods and compositions could be used to create mice possessing the delivered polynucleotide in a specific cell type or cell state.

Single or multicellular non-human organisms, preferably non-human mammals, more preferably mice, can be transfected with the compositions by administering the compositions of the present disclosure to the non-human organism. In one embodiment, the polynucleotide remains episomal and does not stably integrate into the genome of the host organism. In another embodiment, the polynucleotide prevents the expression of a gene of interest. Thus, the expression of the polynucleotide in specific cells of the host can be controlled by the amount of polynucleotide administered to the host.

The transfected non-human organisms have several advantages over traditional transgenic organisms. For example, the transfected organism herein can be produced in less time that traditional transgenic organisms without sexual reproduction. Moreover, the expression of the polynucleotide of interest in the host can be directly regulated by the amount of polynucleotide of interest administered to the host. Dosage controlled expression of a polynucleotide of interest can be correlated to observed phenotypes and changes in the transfected animal. Additionally, inducible expression and/or replication control elements can be included in the polynucleotide of interest to provide inducible and dosage dependent expression and/or replication. Suitable inducible expression and/or replication control elements are known in the art. Furthermore, the effect of genes and gene modifications in specific cell types and states can be studied without affecting the entire cells of the animal.

### E. Methods of Enhancing Mucosal Immunity

It has been established that bPACE particles including antigens, or nucleic acids encoding antigens can be used to provide enhanced mucosal immunity against pathogens.

Natural infection induces the development of tissue-resident mucosal adaptive immunity that act as sentinels at the site of pathogen encounter, including 1) CD8 T cells that can quickly kill infected cells preventing amplification and spread of a pathogen within an infected person; 2) B cells that produce specific mucosal antibodies called secretory IgA that resides at the mucosa to prevent pathogen entry and infection; and 3) CD4 T cells that can help CD8 T cells and B cells to function optimally. Parenteral vaccination given as intramuscular injection (Shots) provides excellent systemic immune protection but does not induce robust mucosal immunity. Mucosal exposure to cognate antigens and/or inflammation is key to the development of tissue-resident memory T and B cell responses.

Methods for inducing or enhancing a robust mucosal immunity to an exogenous antigen in mucosal and epithelial tissues of a subject are provided. The methods effectively generate one or more populations of tissue resident T cells and B cells in the recipient. In preferred embodiments, the methods provide a population of CD8⁺ tissue-resident memory (T_{RM}) cells, CD4⁺ tissue-resident memory (T_{RM}) cells, and/or memory B cells at the mucosal tissue, protective against the vaccinating antigen(s) and/or the original host where the vaccinating antigen is sourced. In one embodiment, methods induce a population of CD69⁺CD8⁺ tissue-resident memory (T_{RM}) cells against the vaccinating antigen(s) in the lung and/or mediastinal lymph node. In one embodiment, methods induce a population of CD69⁺CD103⁺CD8⁺ tissue-resident memory (T_{RM}) cells against the vaccinating antigen(s) in the lung and/or mediastinal lymph node. In a further embodiment, methods induce one or more populations of CXCR5⁺PD1⁺ follicular helper CD4+ T cells (T_{fh} cells), GL7⁺ germinal center B cells, CD138+ antibody-secreting cells (ASC), and antigen-specific B cells in the draining lymph node. In some embodiments, the method provides an effective increase in IgA and/or IgG antibodies against the vaccinating antigen(s) in BALF and/or in the serum.

Methods for inducing or enhancing a robust mucosal immunity to an exogenous antigen in mucosal and epithelial tissues of a subject are provided. The methods effectively generate one or more populations of tissue resident T cells and B cells in the recipient. In preferred embodiments, the methods provide a population of CD8⁺ tissue-resident memory (T_{RM}) cells, CD4⁺ tissue-resident memory (T_{RM}) cells, and/or memory B cells at the mucosal tissue, protective against the vaccinating antigen(s) and/or the original host where the vaccinating antigen is sourced. In one embodiment, methods induce a population of CD69⁺CD8⁺ tissue-resident memory (T_{RM}) cells against the vaccinating antigen(s) in the lung and/or mediastinal lymph node. In one embodiment, methods induce a population of CD69⁺CD103⁺CD8⁺ tissue-resident memory (T_{RM}) cells against the vaccinating antigen(s) in the lung and/or mediastinal lymph node. In a further embodiment, methods induce one or more populations of CXCR5⁺PD1⁺ follicular helper CD4+ T cells (Tₘ cells), GL7⁺ germinal center B cells, CD138+ antibody-secreting cells (ASC), and antigen-specific B cells in the draining lymph node. In some embodiments, the method provides an effective increase in IgA and/or IgG antibodies against the vaccinating antigen(s) in BALF and/or in the serum.

The vaccine composition including polymeric particles and a polynucleotide encoding an antigen can be administered by a variety or routes, typically pulmonary or by injection, but may also be via a mucosal route (gastrointestinal tract including mouth, nasal, gastrointestinal, vaginal, and rectal mucosa). In preferred embodiments, the mucosal tissue is pulmonary and/or nasal mucosa.

The polymeric particles are particularly suited for administration to the nasal or pulmonary system, or administration to other mucosal surfaces. The particular polynucleotide delivered by the particle can be selected by one of skill in the art depending on the condition or disease to be treated. The polynucleotide can be, for example, a gene or cDNA of interest, mRNA, a functional nucleic acid such as an inhibitory RNA, a tRNA, an rRNA, or an expression vector encoding a gene or cDNA of interest, a functional nucleic acid a tRNA, or an rRNA. In some embodiments two or more polynucleotides are administered in combination.

The compositions can be used to provide protective immunity against infectious diseases. In particular, the methods provide protective immunity against one or more respiratory diseases. The diseases for which protective immunity is provided are determined by the antigen that is provided within the compositions for administration. In preferred embodiments, the methods vaccinate a subject against infection with a respiratory viral disease, such as infection with a coronavirus associated with the development of severe acute respiratory syndrome (SARS-Cov-2), or an influenza virus.

Suitable antigens are derived from or raise a protective immune response against one or more respiratory viruses, for example, orthomyxoviruses, paramyxoviruses, coronaviruses, adenoviruses, herpesviruses, and human bocaviruses. In some embodiments, the antigens are derived from, or raises a protective immune response against, one or more of influenza viruses, parainfluenza viruses, measles viruses, mumps viruses, and respiratory syncytial virus (RSV), human metapneumovirus, severe acute respiratory syndrome (SARS) virus, herpes simplex virus (HSV), cytomegalovirus (CMV), Epstein-Barr virus (EBV), and varicella-zoster virus (VZV). In preferred embodiments, the antigen is derived from an influenza virus, a SARS-Cov-2 virus, or RSV. Typically, the polynucleotide encoding the antigen is a deoxyribonucleic acid (DNA) or a ribonucleic acid (RNA) molecule. In preferred embodiments, the polynucleotide encoding the antigen is a messenger RNA (mRNA). In one embodiment, the polynucleotide encoding the antigen is an mRNA including an open reading frame encoding a coronavirus spike protein sequence, or a portion thereof. In some embodiments, the coronavirus is a variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).

An effective amount of the vaccine composition induces a mucosal immune response and/or a long lasting and protective mucosal immunity specific to the antigen in a subject. In some embodiments, the vaccine composition is administered in an amount effective to induce one or more of CD8⁺ tissue-resident memory (T_{RM}) cells, CD4⁺ tissue-resident memory (T_{RM}) cells, and/or memory B cells against the antigen, at the mucosal tissue. In preferred embodiments, the vaccine composition induces an increased number of CD8⁺ tissue-resident memory (T_{RM}) cells, CD4⁺ tissue-resident memory (T_{RM}) cells, and/or memory B cells against the antigen, at the mucosal tissue, compared to a vaccine composition delivered in the absence of poly(amine-co-ester) terpolymers via the same route of administration. In some embodiments, the vaccine composition is administered in an amount effective to induce mucosal Immunoglobulin A (IgA) against the antigen. In preferred embodiments, the vaccine composition induces an increased quantity of mucosal IgA against the antigen compared to a vaccine composition delivered in the absence of bPACE polymeric particles via the same route of administration. Exemplary mucosal IgA is secretory IgA, for example, detected in the mucosal lavage fluid selected from bronchoalveolar lavage fluid, intestinal lavage fluid, gut lavage fluid, and vaginal lavage fluid.

In some embodiments, the method includes the steps of i) administering via a systemic or mucosal route of administration an effective amount of a priming vaccine composition and subsequently ii) administering via a mucosal route to the subject an effective amount of a boosting vaccine composition. This method is based on the discovery that systemic priming of a subject using a vaccine expressing an antigen selectively augments or enhances the induction of antigen-specific mucosal immunity upon subsequent immunization or boosting, via a mucosal route, with a composition containing the same antigen or a fragment thereof.

In one embodiment, the method includes the steps of i) administering via an intramuscular route of administration an effective amount of a priming vaccine composition including mRNA encoding one or more antigens and subsequently ii) administering via a mucosal route to the subject an effective amount of a boosting vaccine composition including mRNA encoding the same antigens to establish tissue-resident immunity.

In another embodiment, the method includes the steps of i) administering via a mucosal route of administration an effective amount of a priming vaccine composition including mRNA encoding one or more antigens and subsequently ii) administering via a mucosal route to the subject an effective amount of a boosting vaccine composition including mRNA encoding the same antigens to establish tissue-resident immunity.

In preferred embodiments, the boosting vaccine composition includes bPACE polymeric particles for effective delivery of mRNA to the mucosal surface. The mucosal immunity is enhanced compared that induced without bPACE polymeric particles, for example, in terms of the number of tissue resident memory T cells, tissue resident memory B cells, and mucosal IgA against one or more vaccinating antigens at a mucosal site, particularly in the respiratory tract.

The boosting composition is administered via a selected mucosa. The preferred route of administration for boosting composition is the intranasal route. Delivery can be accomplished by aerosol, nebulizer, or by depositing a liquid in the nasal cavity.

In some embodiments, the priming includes previous exposure to the antigen via a mucosal route. For example, in some embodiments, the subject is a subject who has previously been exposed to the antigen via administration of an effective amount of a vaccine composition comprising the antigen to mucosal tissue to the subject. Exemplary mucosal tissues include pulmonary, nasal, oral, gastrointestinal, vaginal, and rectal mucosa.

A subject in need of treatment is a subject having or at risk of having an infection (e.g., a subject having or at risk of contracting a viral, bacterial, fungal, or protozoal infection. The methods are particularly suited for those at risk of exposure to one or more respiratory pathogens such as severe acute respiratory syndrome (SARS) virus and influenza virus. The methods effectively induce mucosal immunity including tissue-resident memory T and B cells. In some embodiments, the methods provide protective mucosal and systemic immunity against one or more antigens from an Orthomyxoviridae (e.g., Influenza virus A and B and C), or Coronaviridae (e.g., Coronavirus, such as severe acute respiratory syndrome (SARS) virus).

A subject having an infection is a subject that has been exposed to an infectious microorganism and has acute or chronic detectable levels of the microorganism in his/her body or has signs and symptoms of the infectious microorganism. Methods of assessing and detecting infections in a subject are known by those of ordinary skill in the art. A subject at risk of having an infection is a subject that may be expected to come in contact with an infectious microorganism. Examples of such subjects are medical workers or those traveling to parts of the world where the incidence of infection is high. Therefore, in some embodiments, the subject is an individual who has one or more risk factors including being a medical worker, living in or traveling to parts of the world where the incidence of infection is high, or who has a close contact with an infected individual, for example, is co-habiting or working with one or more infected individuals. In some embodiments, the subject is at an elevated risk of an infection because the subject has one or more physiological risk factors to have an infection. Examples of risk factors to have an infection include, for example, immunosuppression, immunocompromised, age, trauma, burns (e.g., thermal burns), surgery, foreign bodies, cancer, newborns especially newborns born prematurely. Therefore, in some embodiments, the subject is an individual who has one or more risk factors including immunosuppression, immunocompromised, old age, trauma, burns (e.g., thermal burns), surgery, foreign bodies, cancer, newborns, and newborns born prematurely.

The degree of risk of an infection depends on the multitude and the severity or the magnitude of the risk factors that the subject has. Risk charts and prediction algorithms are available for assessing the risk of an infection in a subject based on the presence and severity of risk factors. Other methods of assessing the risk of an infection in a subject are known by those of ordinary skill in the art. In some embodiments, the subject who is at an elevated risk of an infection may be an apparently healthy subject. An apparently healthy subject is a subject who has no signs or symptoms of disease.

Suitable controls are known in the art and include, for example, an untreated subject, or a placebo-treated subject. A typical control is a comparison of a condition or symptom of a subject prior to and after administration of the composition. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art.

In the case of enhancing mucosal immunity, the effect of the polynucleotides-based vaccines associated or encapsulated within branched poly(amine-co-ester) (bPACE) polymeric particles in the form of micellular particles or solid polymeric nanoparticles can be compared to a control. In some embodiments, the control is one having had a priming vaccine composition and/or a boosting vaccine composition via non-mucosal route, or priming and boosting both via intramuscular route. In some embodiments, the control is one having had a priming vaccine composition and/or a boosting vaccine composition without the use of bPACE polymers. In a further embodiment, the control is one having had a priming vaccine composition and a boosting vaccine composition, neither of which uses of bPACE polymers as a vaccine carrier.

### VI. Kits

Kits or packs that supply the elements necessary to conduct transfection of eukaryotic or prokaryotic organisms, in particular the transfection of specific cell types or cell states are disclosed. In accordance with one embodiment, a kit is provided comprising the polymers alone or in combination with nucleic acid agent. The polymer can be combined with a polynucleotide of the user's choosing to form a complex which can be used to transfect a host or a host cell. The polymer can be further mixed with the coating to provide cell-type or cell-state specific tropism. Kits or packs that supply the elements necessary for inducing protective mucosal immunity against one or more pathogenic antigens are also disclosed. In accordance with one embodiment a kit is provided comprising the polymers, and an antigenic polypeptide or a polynucleotide encoding the antigen, form a complex which can be used to deliver the antigen to a target cell. The particle can be further mixed with the coating to provide cell-type or cell-state specific tropism.

The individual components of the kits can be packaged in a variety of containers, e.g., vials, tubes, microtiter well plates, bottles, and the like. Other reagents can be included in separate containers and provided with the kit; e.g., positive control samples, negative control samples, buffers, cell culture media, etc. Preferably, the kits will also include instructions for use.

The present invention will be further understood by reference to the following non-limiting examples.

### EXAMPLES

### Example 1: Synthesis and Modification of PACE and bPACE

### Materials and Methods

### Materials

15-pentadecanolide (PDL), N-methyl diethanolamine (MDEA), sebacic acid (SBA), diphenyl ether, Novozym 435 catalyst (Lipase acrylic resin, Candida antarctica lipase B), bovine serum albumin (BSA), triethanolamine (TEOA), carbonyldiimidazole (CDI), and 1,3-Diamino-2-propanol were purchased from MilliporeSigma (Saint Louis, MO, USA). CleanCap EGFP mRNA and CleanCap FLuc mRNA (5moU) were purchased from TriLink Biotechnologies (San Diego, CA, USA). For cell culture, A549 was purchased from ATCC (Manassas, VA, USA), DME/F-12 (1:1) media was purchased from GE Healthcare Life Science (Logan, UT, USA), fetal bovine serum (FBS) was purchased from R&D Systems (Minneapolis, MN, USA) and gentamicin was purchased from Gibco (Waltham, MA, USA). Pierce BCA Protein Assay were obtained from Thermo Fisher Scientific (Waltham, MA, USA). Glo Lysis Buffer, and the Bright-Glo Luciferase Assay System were purchased from Promega (Madison, WI, USA). Precellys hard tissue lysing tubes were obtained from Bertin Instruments (Montigny-le-Bretonneux, France).

### Synthesis of Branched PACE

The method of synthesis is shown in FIG. 1.

The amount of precursor can be determined depending on the targeted DB and PDL contents. The molar ratio of PDL (x) can be calculated as x=PDL/(PDL+SBA). The molar ratio (y) of dendritic hydroxyl precursor (TEOA) among linear and dendritic hydroxyl precursor (TEOA+MDEA) can be calculated as y=DB/(2−DB), where DB is degree of branching. For example, branched PACE targeting its DB is 0.5 and molar ratio of PDL is 0.1, 2.33 mmol of PDL, 21 mmol of SBA, 10 mmol of MDEA, and 5 mmol of TEOA is needed.

The monomers (MDEA, SBA, PDL, and TEOA) Novozym 435 enzymatic catalyst were transferred to 100 ml-round-flask and dissolved in diphenyl ether.

Prior to reaction, the precursors were dissolved in 60° C. and degassed for 10 min. The monomers were oligomerized for 24 h at 90° C. under 1 atm argon and subsequent condensation reaction for polymerization were followed for 48 h at 90° C. under vacuum (1 mmHg). Purification of polymers was proceeded three times by precipitation and dissolution assisted with n-hexane, dichloromethane, and tetrahydrofuran, followed by removal of residual solvents under vacuum. The characterization of polymers was conducted with 1H-NMR spectrometer (Agilent, DD2 400 MHz) and GPC (Ultimate 3000 UHPLC system, Thermo Fisher Scientific).

### End-Group Modification

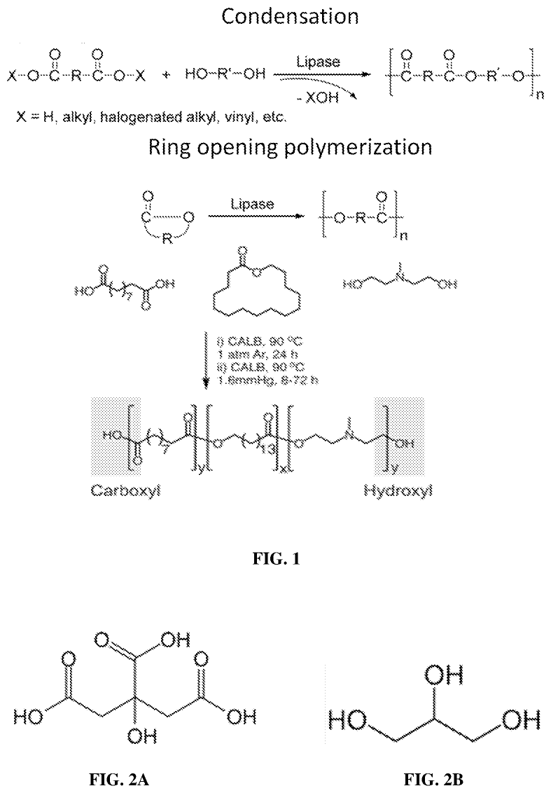

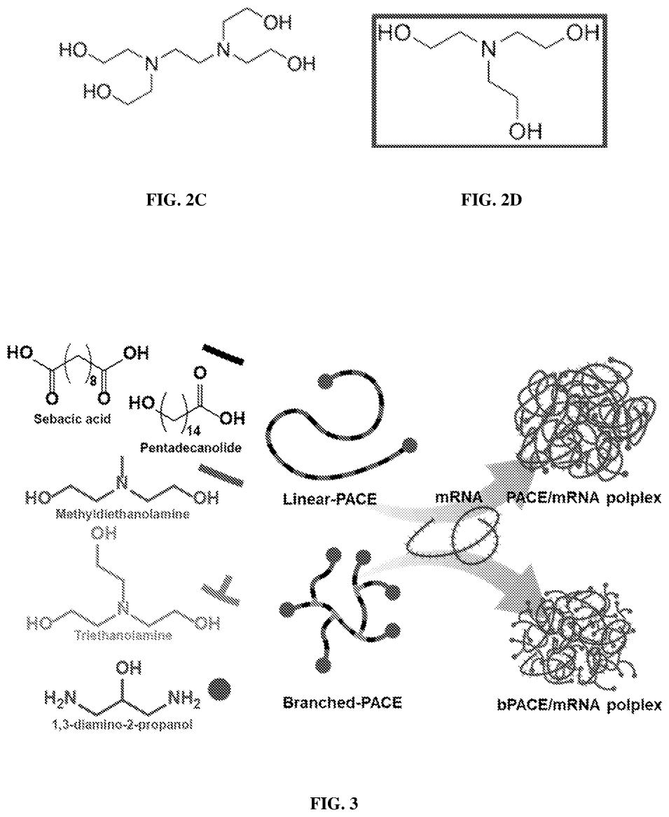

Modification of end-group of bPACE was conducted in two steps, (1) activation of termini of polymer with CDI (FIG. 2) and (2) conjugation of amine to CDI-activated bPACE (bPACE-CDI) (FIG. 3, formerly 5). The terminal group of bPACE was activated using CDI, before end-group modification. Fully dried bPACE was dissolved in anhydrous dichloromethane (DCM) (67 mg/ml), and 6-fold molar excess of CDI compared to terminal groups of polymers was separately dissolved in DCM (72 mg/ml) and added to polymer solution. The reaction proceeded under argon with stirring over 24 hours. The residual CDI and side-products were removed, and bPACE-CDI were collected by extraction using water and DCM (for brine and EA for bPACE whose PDL contents is 0%). After removing water in organic layer with magnesium sulfate, bPACE-CDI was dried under vacuum overnight, and characterized with 1H-NMR spectrometer and GPC. The contents of activated terminal group were calculated with the relative integration of imidazole in 1H-NMR spectroscopy. The 8-fold excessive 1,3-Diamino-2-propanol was dissolved in anhydrous dimethyl sulfoxide (DMSO) and CDI-activated bPACE was dissolved in anhydrous THF. These were mixed with vigorous stirring in reaction vial which was purged with argon.

Followed by overnight reaction, the polymers were collected by precipitation with distilled water and dried overnight.

### Polyplex Formulation

Polymers were fully dissolved 100 mg/mL in DMSO overnight at 37° C. with orbital shaker. To control the weight ratio of polymer to nucleic acid, the various concentration of polyplexes (5, 10, 25, 50 mg/ml) of diluted with DMSO were prepared to maintain amount of DMSO while formation of polyplex. Nucleic acids (mRNA, EGFP) were diluted with sodium acetate buffer (25 mM, pH 5.8) to a final concentration of 20 μg/mL, and polymers were also separately diluted with sodium acetate buffer aiming the target nucleic/polymer ratio at a final concentration of 0.1, 0.2, 0.5, 1, 2 mg/mL and vortexed for 15 s. The diluted polymer solution was transferred to the diluted nucleic acid solution, mixed with 25 sec of vortex. The polyplexes were incubated at room temperature for 10 min before use. Size and ζ-potential of polyplex were measured by dynamic light scattering (DLS, Zetasizer Pro, Malvern).

### In Vitro Transfection

A549 cells were grown with DME/F12(1:1) media supplemented with 10% FBS, 50 μg/mL gentamicin at 37° C., under 5% CO₂. Prior to in vitro transfection experiments, A549 cells were plated in 24-well tissue culture plates at 50,000 cells per well and incubated for 24 h. Cells were treated with prepared polyplex containing 1 μg of EGFP mRNA. Cells were treated for 24. After 24 h incubation, cells transfected with EGFP mRNA were washed once with PBS, dissociated with TrypLE, resuspended in FACS buffer (2% BSA in PBS), and analyzed with flow cytometer (Attune NxT).

### In Vivo Expression

All animal procedures were performed in accordance with the guidelines and policies of the Yale Animal Resource Center (YARC) and approved by the Institutional Animal Care and Use Committee (IACUC) of Yale University. Male BALB/c mice, 7-12 weeks of age, were purchased from The Jackson Laboratory (Bar Harbor, ME, USA). For in vivo experiments, bPACE polyplexes were delivered either by intracheal instillation (IT). Polyplexes were prepared in sodium acetate buffer (25 mM, pH 5.8) to a final concentration of 10 mg/mL polymer and 0.1 mg/mL FLuc mRNA (5moU). For IT delivery, mice were anesthetized under 3% isofluorane (Patterson Veterinary) and suspended by the incisors. The tongue was retracted with tweezers, and 50 μL of the polyplex formulations were administered to the back of the mouth. The tongue was held in the retracted position for the duration of 10 breaths while polyplexes were inhaled. After 24 h, mice were euthanized, and heart perfused with 15 mL PBS. Lung were removed, minced, and transferred to 2 mL Precellys hard tissue lysing tubes with 1 mL Glo Lysis Buffer. Organs were homogenized at 6500 rpm twice for 30 s (Precellys 24) and subsequently centrifuged at 21,000×g for 10 min to remove cell debris. 20 μL of tissue lysates were combined with 100 μL Bright-Glo luciferase substrate and luminescence was measured using an integration time of 10 s (Promega GloMax 20/20). The Pierce BCA Protein Assay Kit was used to measure total protein concentration following the manufacturer's instructions.

### Results

Polymer Design of bPACE

The synthesis of branched PACE (bPACE) is based on the copolymerization of condensation reaction of carboxylates and hydroxyl group and ring-opening reaction assisted with enzymatic catalyst, as shown in the schematic of FIG. 1. As depicted in FIGS. 2A-2D, the branching site of bPACE can be introduced by adding dendritic monomers, whose carboxylate or hydroxyl moieties is more than two, during polymerization. Since the PACE synthesis mainly occurred by condensation reaction of carboxylates and hydroxyls, DB can be controlled by the molar ratio of dendritic monomers during polymerization. The formulation of linear and branched PACE polyplexes including mRNA is depicted in FIG. 3.

One of the main factors affected by DB is ratio of terminal groups, which is crucial to modulate the extent of amine group introduced during end-group modification. The type and ratio of amines in cationic polymers are strongly determining their pKₐ and buffer capacities, contributing stability of nucleic acid and polyplexes, endosomal escape of nucleic acid delivered, and nucleic transfection of polyplexes. The transfection efficiency assisted with branched PACE can be strongly related with the number of end groups in a single polymer. Depending on the molecular weight of PACE and DB, the number of end-groups can be easily controlled.

Thus, it was decided to study the effect of branching of PACE on nucleic transfection with end-group modification. bPACE with carboxylates end group were synthesized and conjugate 1,3-Diamino-2-propanol to the terminal of bPACE via CDI-activation (FIG. 4).

bPACE Synthesis and Characterization

Three different group of bPACE with 0% (bPACE00), 10% (bPACE10), and 20% (bPACE20) of PDL ratio along with various DB from 0.33, 0.5, and 0.66 were synthesized by increasing the feed ratio of branching monomer (Table 6). The bPACE with acid end-group were analyzed with 1H-NMR, to evaluate the molar ratio of branching mononmer, TEOA, degree of branching. As shown in Table 7 and Table 8, the molar ratio of bPACE synthesized were followed the initial feed ratio, confirming the polymerization of branched monomer was not being hindered due to branching, and DB of bPACEs were successfully controlled to each target. Each amine units in bPACE after CDI conjugation was quantified. After CDI-activation, the distinct peak of imidazole in 1H-NMR was observed (7.07, 7.44 and 8.14 ppm) and used to quantify the ratio of terminal groups of bPACE. The molar ratio of terminal amine unit, imidazole, and dendritic amine unit, TEOA, were increased from 10 to 20% and 0 to 13%, respectively, and linear amine unit, MDEA, proportionally decreased from 42 to 12% as DB got higher, in all bPACE group with different PDL contents.

The resulting polymers and characteristics thereof are shown in the following Tables 1-8.

TABLE 1
Monomer feed ratio for synthesis of PACE, PACE-s
and bPACEs and expected Degree of Branching (DB).

| | PDL | MDEA | TEOA | SBA | Novozyme | Diphenyl ether | DB |
|---|---|---|---|---|---|---|---|
| PACE | 2.3 mmol | 20 mmol | 0 mmol | 20 mmol | 0.47 g | 15 g | 0 |
| | (0.56 g) | (0.79 g) | (0 g) | (4.05 g) | | | |
| PACE-s | 2.3 mmol | 20 mmol | 0 mmol | 21 mmol | 0.47 g | 15 g | 0 |
| | (0.56 g) | 0.79 (g) | (0 g) | (4.25 g) | | | |
| bPACE-1 | 2.3 mmol | 16 mmol | 2 mmol | 21 mmol | 0.47 g | 15 g | 0.2 |
| | (0.56 g) | (1.19 g) | (0.75 g) | (4.25 g) | | | |
| bPACE-2 | 2.3 mmol | 13 mmol | 3.3 mmol | 21 mmol | 0.47 g | 15 g | 0.33 |
| | (0.56 g) | (1.59 g) | (0.5 g) | (4.25 g) | | | |
| bPACE-3 | 2.3 mmol | 10 mmol | 5 mmol | 21 mmol | 0.47 g | 15 g | 0.5 |
| | (0.56 g) | (1.19 G0 | (0.75 g) | (4.25 g) | | | |
| bPACE-4 | 2.3 mmol | 10 mmol | 5 mmol | 21 mmol | 0.47 g | 15 g | 0.5 |
| | (0.56 g) | (1.19 g) | (0.75 g) | (4.25 g) | | | |
| bPACE-5 | 2.3 mmol | 6.7 mmol | 6.7 mmol | 21 mmol | 0.47 g | 15 g | 0.67 |
| | (0.56 g) | (0.79 g) | (0.99 g) | (4.25 g) | | | |

TABLE 2
Molar ratio of monomers and expected Degree of Branching
of carboxylate-terminated PACE, PACE-s and bPACEs

| | | | | | | PDL/(PDL + |
|---|---|---|---|---|---|---|
| | PLD | MDEA | TEOA | SBA | DB | SBA) |
| PACE | 5.3% | 47.4% | 0.0% | 47.4% | 0.00 | 10.0% |
| PACE-s | 5.6% | 43.6% | 0.0% | 50.8% | 0.00 | 10.0% |
| bPACE-1 | 5.5% | 42.5% | 2.4% | 49.6% | 0.10 | 10.0% |
| bPACE-2 | 5.6% | 38.7% | 4.8% | 50.8% | 0.20 | 10.0% |
| bPACE-3 | 5.8% | 33.3% | 8.3% | 52.5% | 0.33 | 10.0% |
| bPACE-4 | 6.1% | 26.1% | 13.0% | 54.8% | 0.50 | 10.0% |
| bPACE-5 | 6.4% | 18.2% | 18.2% | 57.3% | 0.67 | 10.0% |

TABLE 3
Monomer ratio and Degree of Branching of carboxylate-terminated
PACE, PACE-s and bPACEs analyzed with ¹H-NMR

| | | | | | | PDL/(PDL + |
|---|---|---|---|---|---|---|
| | PLD | MDEA | TEOA | SBA | DB | SBA) |
| PACE | 6.2% | 44.1% | 0.0% | 49.7% | 0.00 | 11.1% |
| PACE-s | 6.1% | 42.2% | 0.0% | 51.7% | 0.00 | 10.5% |
| bPACE-1 | 6.2% | 39.3% | 3.0% | 51.5% | 0.13 | 10.8% |
| bPACE-2 | 6.6% | 35.7% | 6.3% | 51.3% | 0.26 | 11.4% |
| bPACE-3 | 6.1% | 30.4% | 9.7% | 53.8% | 0.39 | 10.2% |
| bPACE-4 | 6.2% | 24.6% | 14.2% | 55.1% | 0.54 | 10.1% |
| bPACE-5 | 6.8% | 18.1% | 18.7% | 56.4% | 0.67 | 10.7% |

TABLE 4
Monomer ratio and Degree of Branching of end-group modified
PACE, PACE-s and bPACEs analyzed with ¹H-NMR

| | Moiety | | | | |
|---|---|---|---|---|---|
| | | | | | Secondary |
| | | | | Primary | amine |
| | Tertiary amine | | | amine | (Amide) |
| Monomer | MDEA | TEOA | Total | 1,3-diamino-2-propanol | |
| PACE | 65.3% | 0.0% | 65.3% | 17.3% | 17.3% |
| PACE-s | 42.9% | 0.0% | 42.9% | 28.5% | 28.5% |
| bPACE-1 | 56.8% | 3.9% | 60.7% | 19.6% | 19.6% |
| bPACE-2 | 50.2% | 7.6% | 57.8% | 21.2% | 21.1% |
| bPACE-3 | 44.7% | 12.1% | 56.9% | 21.6% | 21.6% |
| bPACE-4 | 32.6% | 15.0% | 47.6% | 26.2% | 26.2% |
| bPACE-5 | 23.6% | 21.6% | 45.15 | 27.4% | 27.4% |

TABLE 5
Ratio of amines groups in end-group modified PACE, PACE-s and bPACEs.

| | | | | | 1,3-diamino- | | PDL/(PDL + |
|---|---|---|---|---|---|---|---|
| | PLD | MDEA | TEOA | SBA | 2-propanol | DB | SBA) |
| PACE | 12.6% | 37.6% | 0.0% | 39.8% | 10.0% | 0.00 | 24.0% |
| PACE-s | 4.5% | 32.0% | 0.0% | 42.0% | 21.2% | 0.00 | 9.7% |
| bPACE-1 | 4.8% | 33.2% | 2.3% | 48.2% | 11.5% | 0.12 | 9.1% |
| bPACE-2 | 5.5% | 30.8% | 4.7% | 46.1% | 13.0% | 0.23 | 10.7% |
| bPACE-3 | 5.4% | 27.1% | 7.4% | 47.1% | 13.1% | 0.35 | 10.2% |
| bPACE-4 | 5.7% | 21.2% | 9.7% | 46.3% | 17.0% | 0.48 | 11.0% |
| bPACE-5 | 5.9% | 15.1% | 13.8% | 47.6% | 17.6% | 0.65 | 11.1% |

TABLE 6
Monomer amount for bPACE synthesis

| | PDL | MDEA | TEOA | SBA | Novozyme | Diphenyl | | PDL/(PDL + |
|---|---|---|---|---|---|---|---|---|
| Polymer | (mmol) | (mmol) | (mmol) | (mmol) | (g) | ether (g) | DB | SBA) |
| bPACE01 | — | 13.33 | 3.33 | 21 | 0.47 | 14 | 0.33 | 0 |
| bPACE02 | — | 10 | 5 | 21 | 0.47 | 14 | 0.5 | 0 |
| bPACE03 | — | 6.67 | 6.67 | 21 | 0.47 | 14 | 0.67 | 0 |
| PACE10 | 2.33 | 20 | — | 21 | 0.47 | 15 | 0 | 10% |
| bPACE11 | 2.33 | 13.33 | 3.33 | 21 | 0.47 | 15 | 0.33 | 10% |
| bPACE12 | 2.33 | 10 | 5 | 21 | 0.47 | 15 | 0.5 | 10% |
| bPACE13 | 2.33 | 6.67 | 6.67 | 21 | 0.47 | 15 | 0.67 | 10% |
| bPACE21 | 3.93 | 10 | 2.5 | 15.75 | 0.41 | 15 | 0.33 | 20% |
| bPACE22 | 3.93 | 7.5 | 3.75 | 15.75 | 0.41 | 15 | 0.5 | 20% |
| bPACE23 | 3.93 | 5 | 5 | 15.75 | 0.41 | 15 | 0.67 | 20% |

TABLE 7
Molar ratio of bPACE in initial reactant of bPACE synthesis

| | PDL | MDEA | TEOA | SBA | | PDL/(PDL + |
|---|---|---|---|---|---|---|
| Polymer | (mol %) | (mol %) | (mol %) | (mol %) | DB | SBA) |
| bPACE01 | — | 35.4 | 8.8 | 55.8 | 0.33 | 0 |
| bPACE02 | — | 27.8 | 13.9 | 58.3 | 0.50 | 0 |
| bPACE03 | — | 19.4 | 19.4 | 61.2 | 0.67 | 0 |
| bPACE11 | 5.8 | 33.3 | 8.3 | 52.5 | 0.33 | 10% |
| bPACE12 | 6.1 | 26.1 | 13.0 | 54.8 | 0.50 | 10% |
| bPACE13 | 6.4 | 18.2 | 18.2 | 57.3 | 0.67 | 10% |
| bPACE21 | 12.2 | 31.1 | 7.8 | 48.9 | 0.33 | 20% |
| bPACE22 | 12.7 | 24.2 | 12.1 | 50.9 | 0.50 | 20% |
| bPACE23 | 13.2 | 16.8 | 16.8 | 53.1 | 0.67 | 20% |

TABLE 8
Molar ratio of synthesized bPACE

| | PDL | MDEA | TEOA | SBA | | PDL/(PDL + |
|---|---|---|---|---|---|---|
| Polymer | (mol %) | (mol %) | (mol %) | (mol %) | DB | SBA) |
| bPACE01 | — | 32.8 | 9.2 | 58.1 | 0.36 | 0 |
| bPACE02 | — | 25.5 | 13.9 | 60.6 | 0.52 | 0 |
| bPACE03 | — | 18.1 | 18.9 | 63.1 | 0.68 | 0 |
| bPACE11 | 7.5 | 31.2 | 8.5 | 52.8 | 0.35 | 12% |
| bPACE12 | 6.5 | 24.9 | 11.3 | 57.3 | 0.48 | 10% |
| bPACE13 | 7.9 | 16.4 | 17.0 | 58.7 | 0.67 | 12% |
| bPACE21 | 14.8 | 27.6 | 9.0 | 48.5 | 0.40 | 23% |
| bPACE22 | 15.8 | 23.2 | 11.6 | 49.3 | 0.50 | 24% |
| bPACE23 | 16.2 | 17.7 | 15.7 | 50.5 | 0.64 | 24% |

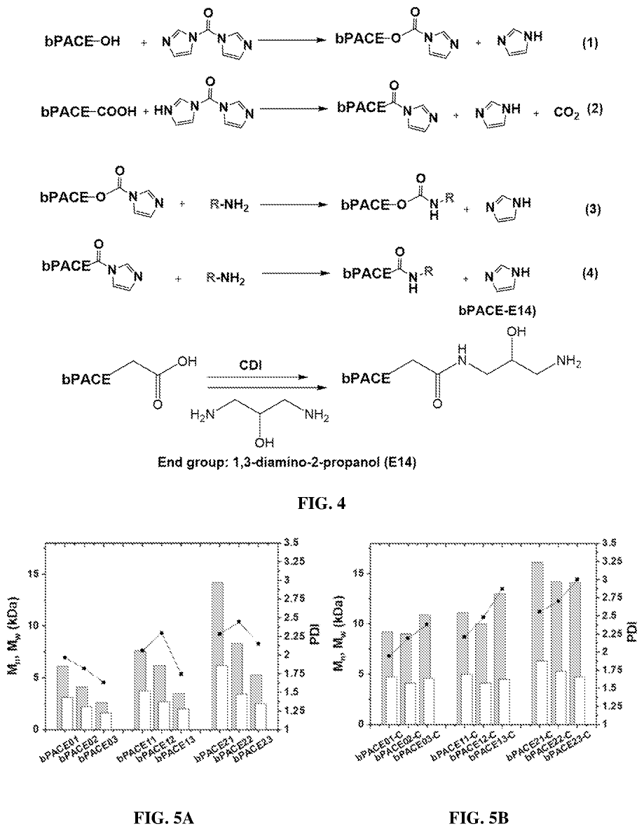

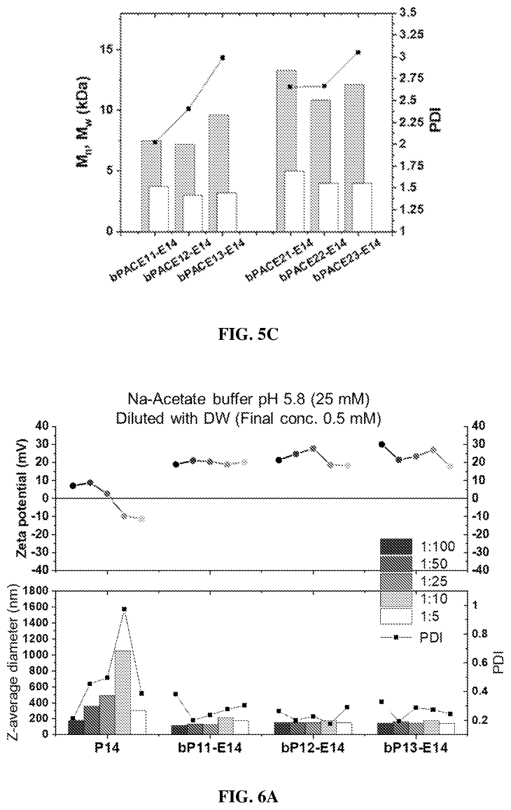

Gel permeation chromatography (GPC), shows that the molecular weights of acid-end bPACE were affected by DB. Both number-averaged molecular weight (Mₙ) and weight-averaged molecular weight (M_{w}) were tended to be reduced by increasing DB in all groups (FIGS. 5A-5C). This is possibly because the electrostatic interaction of carboxylate ends interacting with amine groups inside of polymers induced the shrinkage of the branched PACE. However, all CDI-activated bPACE showed similar molecular weights regardless of their DB. Especially the molecular weight CDI-activated bPACE with higher DB (bPACE03, bPACE13, and bPACE23) increased the most and approached to molecular weight of bPACE with smaller DB, which evidences that the observation of smaller of M_{w} were attributed by interaction of acid terminal groups and cationic amine groups in back-bone.

Formation of bPACE Polyplexes

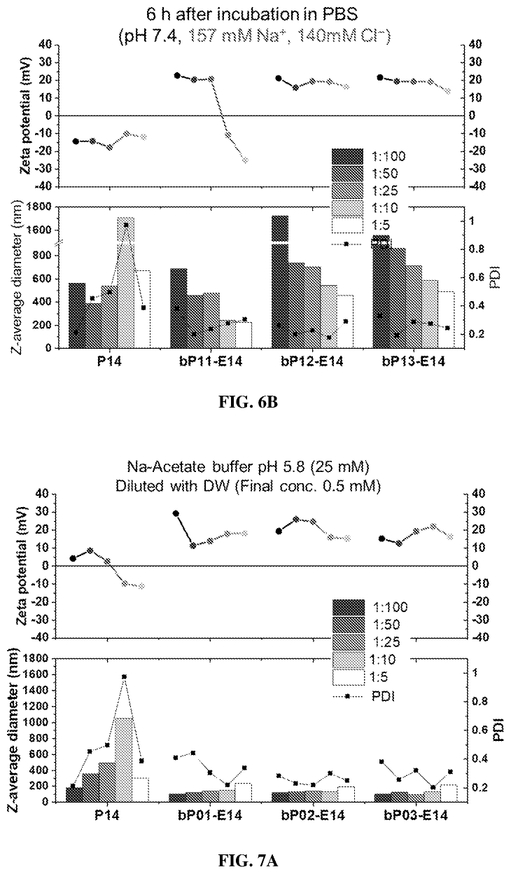

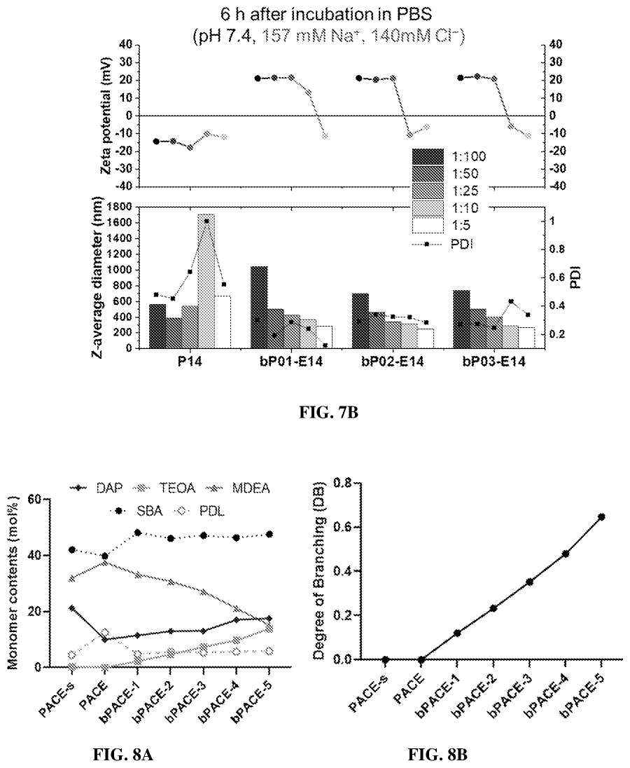

Previous studies on end-group modified cationic polymers showed that the encapsulation of nucleic acid and their stabilities dominated by type of end-groups. The additional amines on the terminal groups by end-group modification increases the amine contents in single polymers, and the extent of terminal amines has been increased with branched structures. Moreover, previous studies on the branched cationic polymers showed that the branched structure enhanced the colloidal stability of polyplex assembled compared with linear counterpart. To evaluate the nucleic acid condensation efficiency and stability of polyplexes depending on the bPACE, mRNA-bPACE polyplexes were formulated with various weight ratio of polymer/mRNA weight ratio from 100:1 to 5:1 and characterized their size and surface potential (FIGS. 6A-7B).

bPACE polyplexes and linear PACE with highest polymer/mRNA ratio (100:1, w/w) were in a similar size range as measured by DLS (120-200 nm) and their ζ-potentials were exhibit positive surface potential (10-30 mV). By reducing ratio the ratio of polymer to mRNA, the ζ-potentials of polyplexes formulated with linear PACE (P14) got decreased. Since the colloidal stability of polyplex are mostly contributed by electrostatic repulsion, the size and PDI of the linear PACE polyplex got larger as the surface potentials approached 0 and restored its size and PDI when the surface potentials inverted to negative (−15 mV). In contrast, the sizes and surface potentials of all polyplexes formulated with bPACE were maintained to be small (<200 nm) and positive (20 mV) even to 5:1 of polymer/mRNA ratio (FIGS. 6A and 7A).

To evaluate the colloidal stability of bPACE polyplexes in physiological media, the size and surface potentials were analyzed after incubating in PBS (pH 7.4) for 6 hours (FIGS. 6B and 7B). The size of polyplexes were increased in all group, mainly due to higher salt concentration (140 mM of [NaCl]). However, there were remarkable differences in the change of polyplexes surface charge between linear PACE and branched PACE. The surface charge of polyplexes with linear PACE were changed to negative despite of highest polymer/mRNA ratio. However, the surface potential of polyplexes maintained mostly positively with bPACE. The similar charge inversion was observed when the polymer/mRNA ratio and DB is small. For bPACE with 10% PDL group (bPACE10 group), negative potential was only observed from the bPACE polyplexes with smallest DB (bPACE11) when the polyplexes were formulated with the polymer/mRNA ratio of 10:1 and 5:1 (FIG. 6B). For bPACE polyplexes without PDL (bPACE00 group), polyplexes formulated with 5:1 polymer/mRNA ratio showed negative ζ-potential (−10 mV) in all samples, and polyplexes with 10:1 ratio also found to be negative potential when DB is smaller than 0.5 (bPACE01 and bPACE12). This indicates that branched structure contributed the stable positive surface potential of polyplexes to be preserved despite larger amount of mRNA incorporation, suggesting that the lower extent of branched PACE can be enough to transfect mRNA than linear PACE.

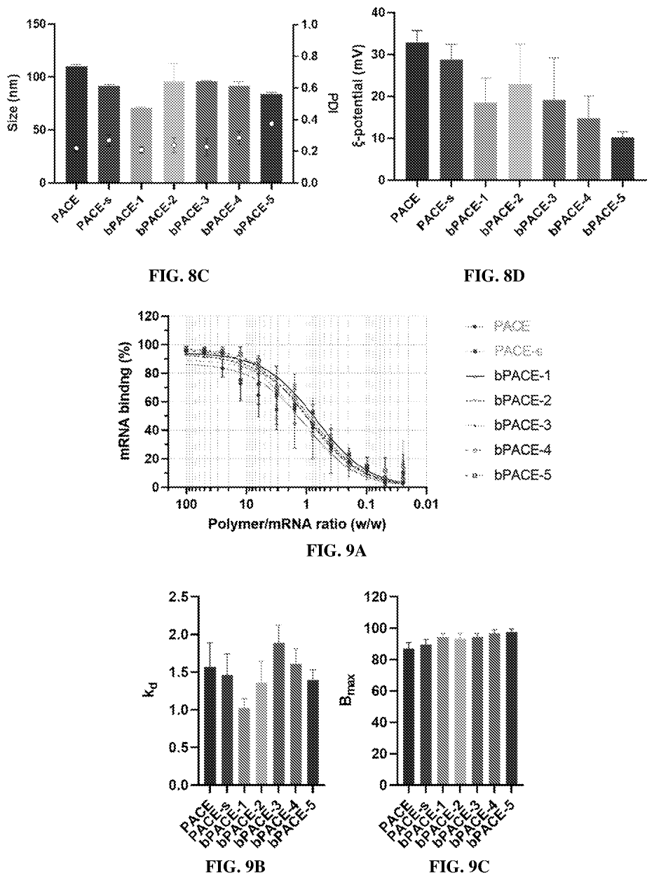

In addition, corresponding stability and biophysical characterization was carried out for polyplexes formulated using each of the PACE, PACE-short (PACE-s), bPACE-1, bPACE-2, bPACE-3, bPACE-4, and bPACE-5 based on Tables 1-5. The Monomer contents of bPACEs and PACE, degree of branching (DB) of bPACEs, Hydrodynamic size (z-average) and polydispersity index (PDI) and Zeta-potential of polyplexes formulated with bPACE was determined to vary in a controllable and tunable fashion (see FIGS. 8A-8D).

Physicochemical properties of the bPACE formulations (bPACE-1,2,3,4,5) were assessed and compared with PACE. mRNA release profile after treating heparin to mRNA-bPACE polyplexes was visualized and quantitated using Ribogreen assay, mRNA were dissociated from polyplexes with heparin/mRNA to mRNA ratio with 20:1 (w/w), and the relative amount of released mRNA from polyplexes after 1 minute of various amount of heparin added, and after 10 minute of various amount of heparin added were tested.

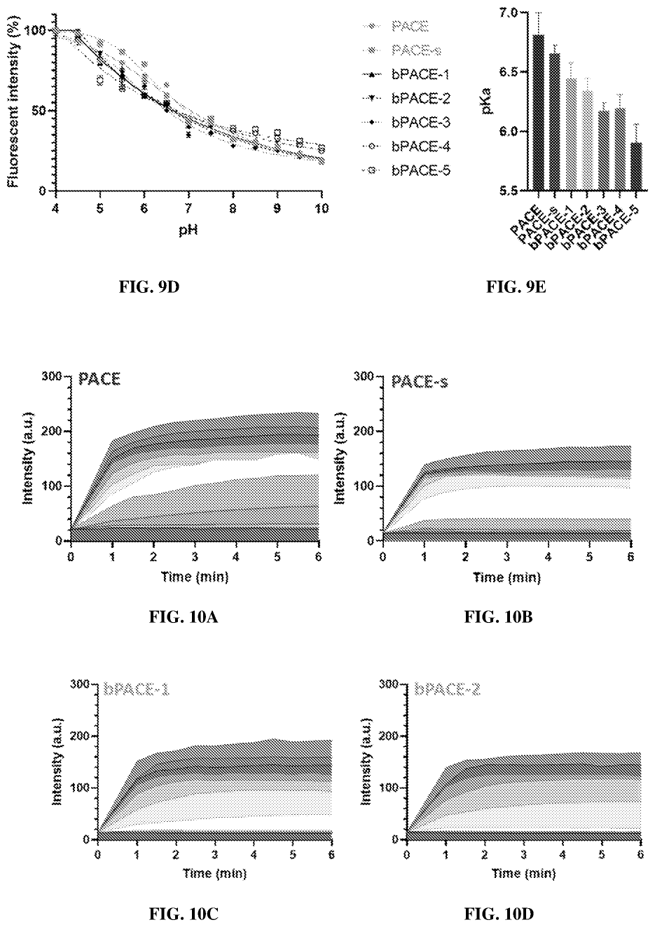

Evaluation of mRNA loading of PACE, PACE-s and bPACEs was determined using a Ribogreen assay using multiple different polymer to mRNA ratios (w/w). The non-linear curve fit of the Ribogreen assay for mRNA-polymer binding, as determined using a one-site binding model for polyplexes formulated using each of the PACE, PACE-s, bPACE-1, bPACE-2, bPACE-3, bPACE-4, and bPACE-5 is depicted in FIG. 9A. Determination of dissociation constant (kd) obtained from the curve fit for each polyplex is depicted in FIG. 9B, and maximum specific binding (Bₘₐₓ), is depicted in FIG. 9C.

TNS (6-(p-Toluidino)-2-naphthalenesulfonyl chloride) binding assay was then carried out to determine pKa values for each bPACE1-5 polyplex. Fluorescent intensity of TNS was calculated at various pH, and curves were determined with asymmetric sigmodal non-linear fitting. pKa values of polyplexes were obtained from the curve fit (see FIGS. 9D-9E).

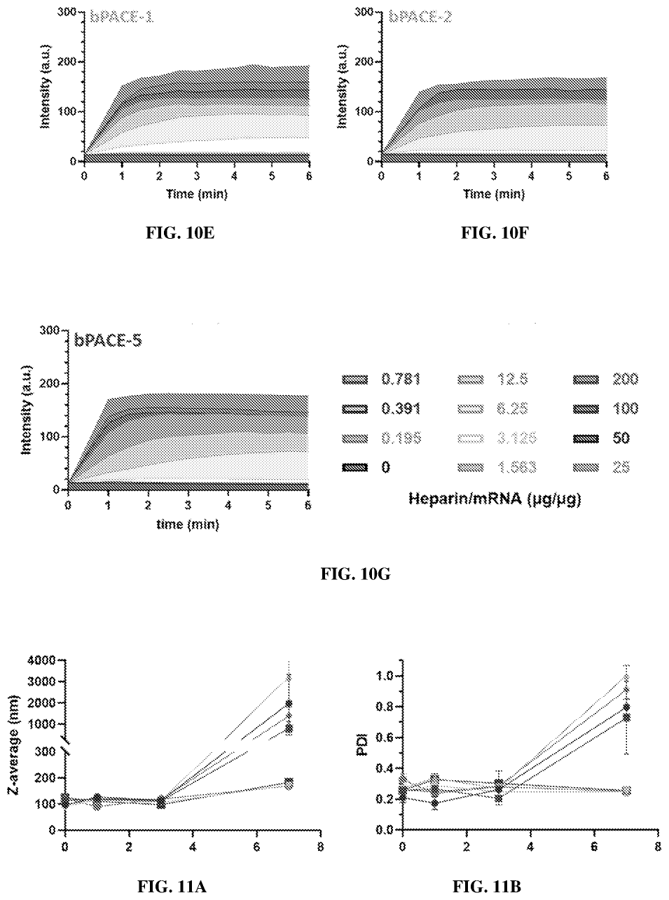

mRNA release profiles were determined after treatment using heparin. Each of the mRNA-bPACE polyplexes (PACE, PACE-s, bPACE-1, bPACE-2, bPACE-3, bPACE-4, and bPACE-5) was analyzed for mRNA release over 6 minutes upon exposure to various heparin/mRNA to mRNA ratios, analyzed by Ribogreen assay (see FIGS. 10A-10G).

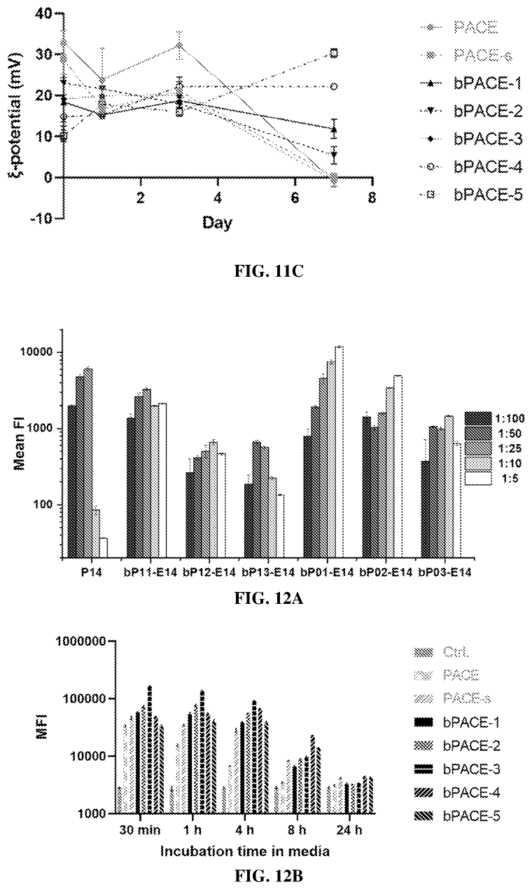

In addition, colloidal Stability of PACE- or bPACE-polyplexes was also assessed. Changes of hydrodynamic size, PDI, zeta-potential of polyplexes stored at room temperature (22° C.) was assayed over time, as depicted in FIGS. 11A-11C.

### Example 2: In Vitro Transfection Assay of bPACE Polyplex

The aim of polymeric vector for nucleic acid delivery is for maximizing expression efficiency while minimizing toxicity. However, the administration of cationic polymer with higher concentration improves transfection efficiency but it costs higher toxicity. Although the reducing the dose of cationic polymer would be beneficial, the lack of protecting capability of nucleic acid with the limited polymer to nucleic acid results poor transfection. Since the polyplex formed by bPACE were able to maintain their stability possessing positive surface potential even with polymer to mRNA ratio, it is expected to be beneficial to improve transfection in vitro and in vivo while reducing dose of polymeric vector.

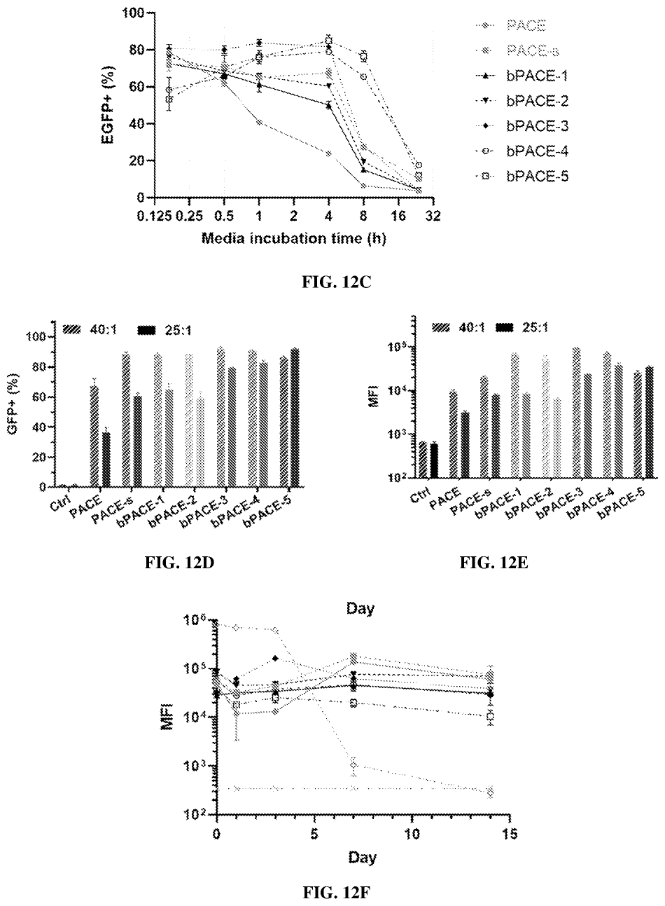

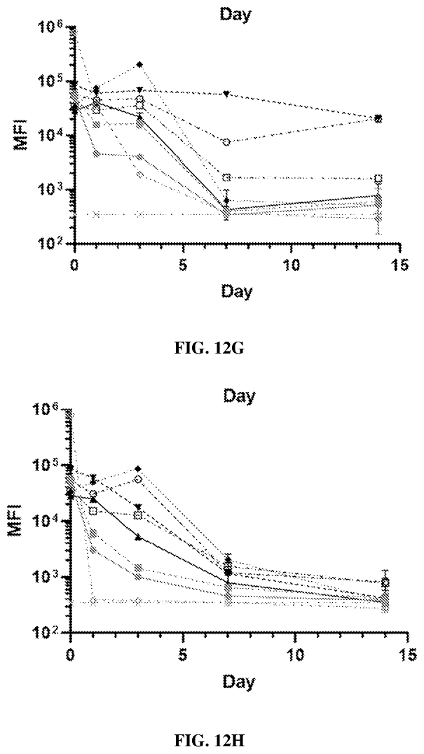

In vitro transfection of mRNA using bPACE polyplex was evaluated with A549 cell, which is lung epithelial cell line. Cells were treated EGFP loaded polyplexes formulated with various polymer/mRNA ratio from 100:1 to 5:1 (FIG. 12A).

In addition, the Stability of EGFP/bPACE polyplexes formulated using each of the PACE, PACE-s, bPACE-1, bPACE-2, bPACE-3, bPACE-4, and bPACE-5 in serum containing media was determined in terms of transfection ability. Briefly, mean fluorescence intensity of EGFP signals from EGFP/polyplex-treated A549 cells was determined as varying according to incubation time in serum containing media. Data were quantitated as a percentage of EGFP positive A549 cells treated with EGFP/polyplex-treated which incubated in serum containing media (see FIGS. 12B-12C).

EGFP expression of A549 cells treated with EGFP-mRNA/bPACE polyplexes (each of PACE, PACE-s, bPACE-1, bPACE-2, bPACE-3, bPACE-4, and bPACE-5) formulated with 1:40 and 1:25 of mRNA:polymer ratio, was determined by flow cytometry. Fluorescence intensity (EGFP) of A549 cells treated with 1:40 mRNA/polymer ratio and with 1:25 mRNA/polymer ratio was calculated as a percentage of EGFP-expressing cells, and as the Mean fluorescence intensities (see FIGS. 12D-12E).

Polyplexes formulated using each of the PACE, PACE-s, bPACE-1, bPACE-2, bPACE-3, bPACE-4, and bPACE-5 were formulated with 1:25 mRNA to polymer ratio (w/w), and percentages of EGFP expressed by A549 cells transfected with polyplexes or lipofectamine MessengerMax (LipoMM) stored at 4° C., 22° C., and 37° C. was observed and Mean fluorescence intensity of EGFP signal transfected A549 cells with polyplexes or LipoMM stored at 4° C., 22° C., and 37° C. over time was quantified as analyzed by flow cytometry (see FIGS. 12F-12H).

As the stability and surface potentials of bPACE polyplexes were maintained with lower polymer ratio, the GFP expression level was able to be observed with lower bPACE concentration than linear PACE. There were significant higher expression levels with 10:1 or 5:1 polymer ratio of bPACE, but GFP expression was dramatically decreased in 10:1 or 5:1 ratio of linear PACE (P14). The optimal polymer ratio for highest expression level was varied depending on the bPACE. The transfection level of mRNA tended to be decreased by increasing DB. However, bP01-E14, which have least branched without PDL, showed surprisingly higher expression level than any concentration of linear PACE, even with smallest polymer/mRNA ratio (5:1). This suggests that the transfection of mRNA with bPACE can be achieved with lower concentration of cationic polymer, and it is anticipated that less dose of polymers is required for nucleic acid delivery.

### Example 3: In Vivo mRNA Transfection with bPACE

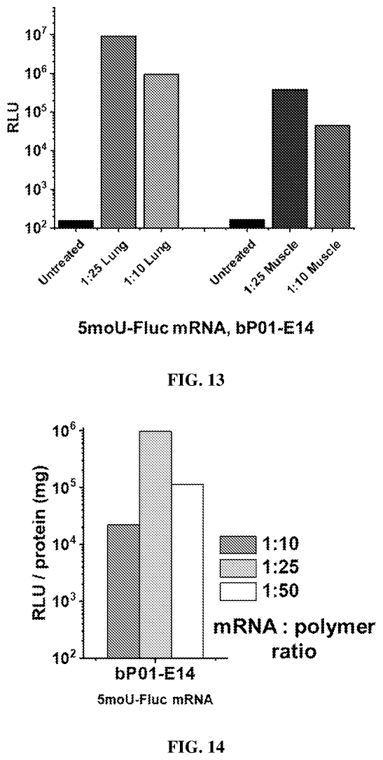

To demonstrate the transfection of mRNA in a low range of polymer/mRNA ratio, 5moU-Fluc mRNA was delivered using bP01-E14 polyplex. bP01-E14 polyplexes were formulated with 25:1 and 10:1 polymer/mRNA ratio and administrated to mice by either intratracheal (IT) or intramuscular (IM) delivery (see FIG. 13).

Compared to the in vitro transfection assay, the optimal polymer ratio was determined by normalization with protein extent in the tissue using the microBCA assay.

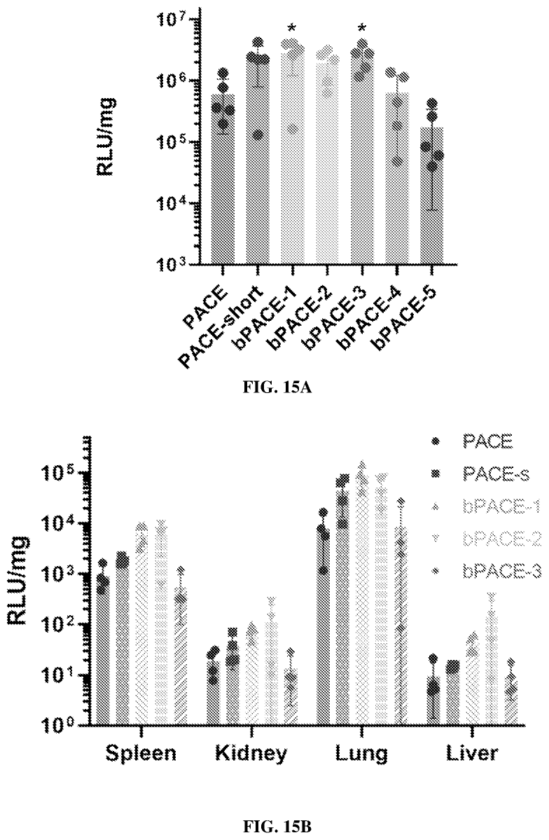

In addition, luciferase (FLuc) mRNA was delivered in vivo using bPACE polyplexes (formulated using each of the PACE, PACE-s, bPACE-1, bPACE-2, bPACE-3, bPACE-4, and bPACE-5) to test animals via intratracheal (IT) delivery to the lung, or by systemically by intravenous (IV) administration. Luciferase expression in tissue was quantified by luminescence assay for IT delivery (lungs) and IV delivery (lung, spleen, liver, kidney), as depicted in FIGS. 15A-15B.

Complete blood count analysis was carried out 7 days after intrathecal administration of polyplexes formulated with 1:25 mRNA to polymer ratio; histological analyses of the lungs were carried out at day 8 after intratracheal administration via H&E staining, using 40× magnification for visualization of cellular architecture in the alveoli and bronchi.

Twenty-four hours after administration, transfection was observed both in the lung and muscle tissue. There was 10 times brighter luminescence signal from tissue injected polyplexes with ratio of 25:1 than 10:1 ratio. It was found that 25:1 ratio was the optimal ratio for mRNA transfection in vivo, and the luminescence signal was high over 10⁶ RLU/mg (FIG. 14).

There were no adverse effects upon the blood, as determined by complete blood count analyses in comparison with untreated control samples.

Histological analyses of the lungs after administration (Day 8) indicated that alveolar architecture and bronchiolar architecture was maintained in all treated samples (i.e., control, vs. PACE and bPACE/bPACE 1-5).

Those skilled in the art will recognize, or be able to ascertain using no more than routine experimentation, many equivalents to the specific embodiments of the invention described herein. Such equivalents are intended to be encompassed by the following claims.

1. A branched poly(amine-co-ester) (bPACE polymer) having a structure: 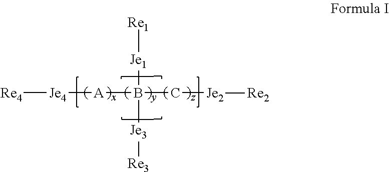
wherein:
Re1, Re2, Re3, and Re4 are independently absent or chemical entities containing a hydroxyl group, a primary amine group, a secondary amine group, a tertiary amine group, or combinations thereof, Je1, Je2, Je3, and Je4 are independently absent, —O—, —S—, —NH—, NRg, —NHC(O)—, —C(O)NH—, —OC(O)—, —C(O)O—, —NRgC(O)—, —C(O)NRg—, —OC(O)NH—, —NHC(O)O—, —OC(O)NRg—, —NRgC(O)O—, —NHC(O)NH—, —NRgC(O)NH—, —NHC(O)NRg—, —NRgC(O)NRg—, —SO—, or —S(O)₂—, wherein Rg is, independently for each occurrence, an alkyl, cycloalkyl, heterocycloalkyl, alkylaryl, alkenyl, alkynyl, aryl, or heteroaryl group, optionally substituted with between one and five substituents independently selected from alkyl, cyclopropyl, cyclobutyl ether, amine, halogen, hydroxyl, thiol, ether, thioether, nitrile, CF₃, ester, amide, urea, carbamate, thioether, carboxylic acid, and aryl,
x, y, and z are independently integers from 1 to 1,000,
A, B, and C, are, independently for each occurrence, Formula II: 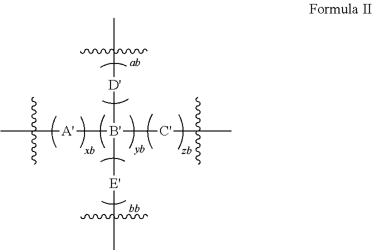
wherein:
xb and zb are independently integers from 0 to 1,000, with the proviso that xb+zb is at least 1,
yb is independently an integer from 1 to 1,000, ab and bb are independently integers from 0 to 1,000, with the proviso that ab+bb is at least 1, in at least one of A, B, and C,
A′ and C′, independently for each occurrence, contain a structure selected from: 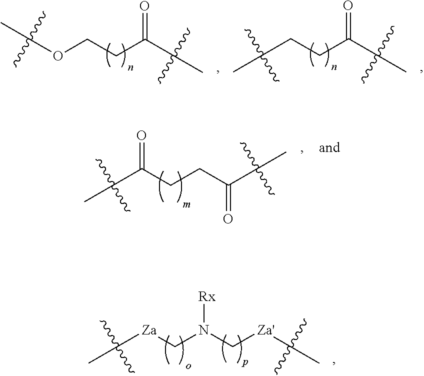
with the proviso that the bPACE contains the structure: 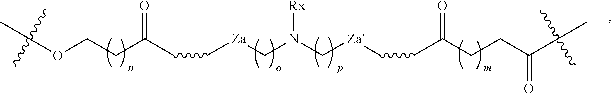
wherein k is 0 or 1,
D′ and E′, independently for each occurrence, contain a structure selected from: 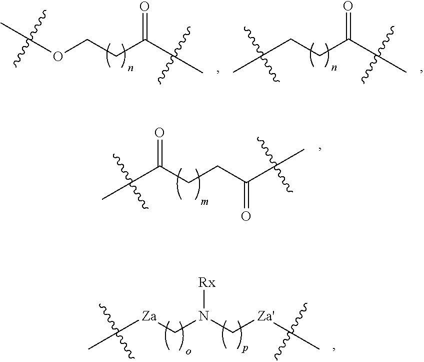
and B′,
each n is independently an integer from 1-30, m, o, and p are independently integers from 1-20, Za and Za′ are independently O or NR′, wherein Rx is hydrogen, substituted or unsubstituted alkyl, or substituted or unsubstituted aryl, or substituted or unsubstituted alkoxy, wherein R′ is hydrogen, substituted or unsubstituted alkyl, or substituted or unsubstituted aryl, and B′, independently for each occurrence, contains a branch point.

2. The bPACE polymer of claim 1, comprising a molar ratio of lactone unit to (lactone unit+diester/diacid) between about 0 and about 0.4.

3. The bPACE polymer of claim 1, wherein:
(i) Rx is hydrogen, substituted or unsubstituted alkyl, or substituted or unsubstituted aryl,
(ii) Za and Za′ are O,
(iii) n is independently an integer from 1 to 24,
(iv) m is an integer from 1 to 10, or
(v) n is 13, m is 7, Rx is unsubstituted alkyl, Za and Za′ are O, and o and p are 2.

4. (canceled)

5. (canceled)

6. (canceled)

7. (canceled)

8. The bPACE polymer of claim 1, wherein B′, independently for each occurrence, comprises a structure selected from: 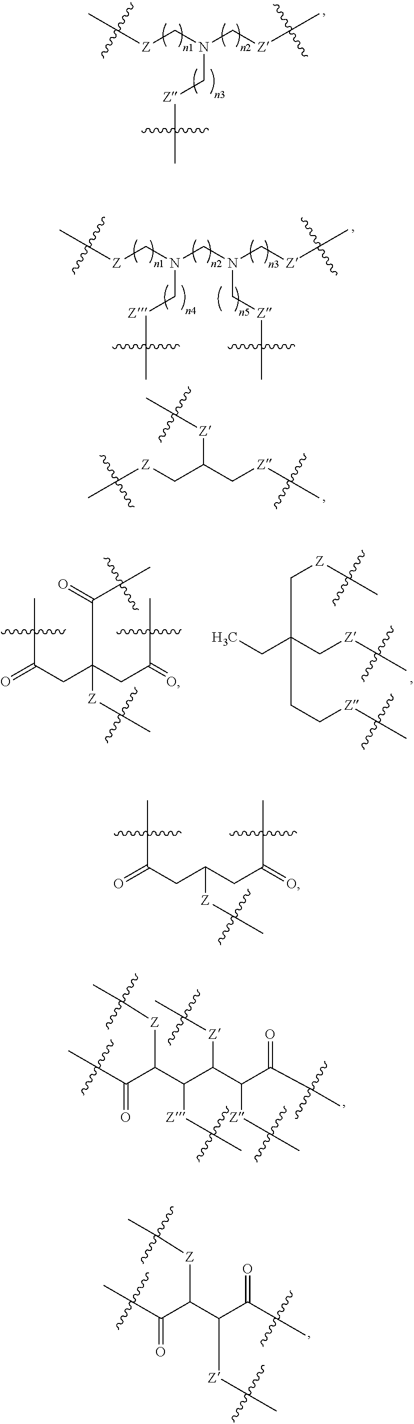 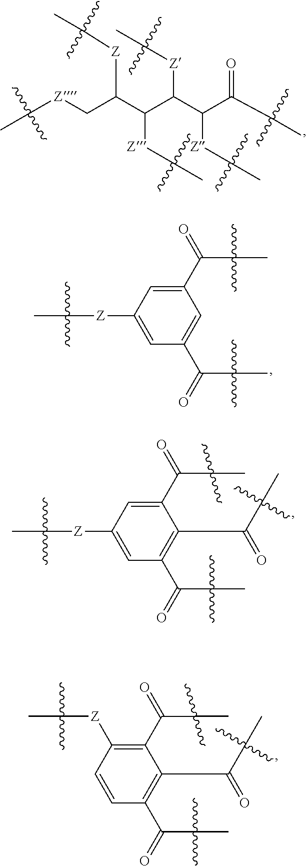
and a combination thereof, wherein each Z, Z′, Z″, Z′″, and Z″″ is independently O, S, or NRy, wherein Ry is hydrogen, substituted or unsubstituted alkyl, substituted or unsubstituted aryl, and
each n1, n2, n3, n4, and n5 is independently an integer from 1 to 20.

9. The bPACE polymer of claim 8, wherein B′, independently for each occurrence, comprises a structure selected from: 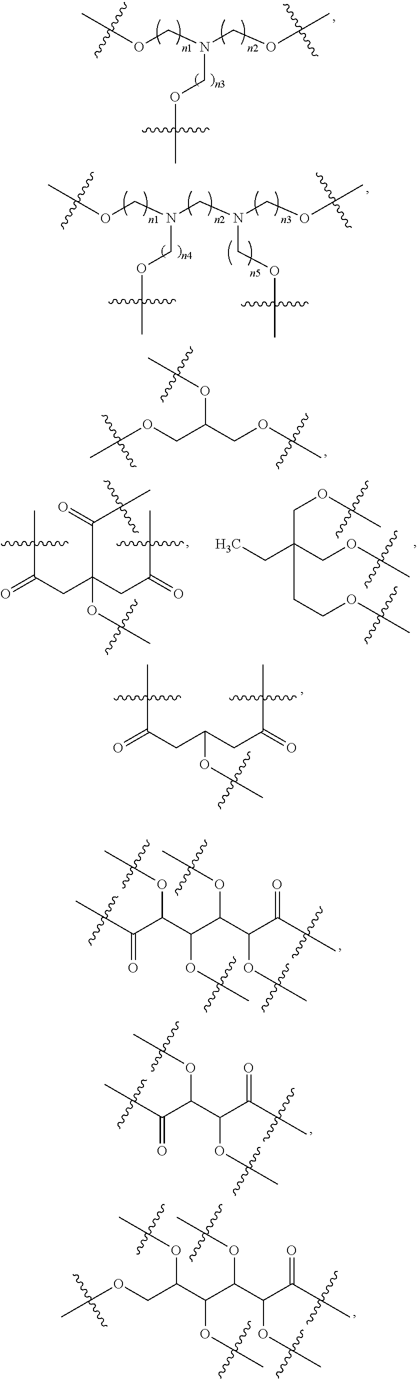 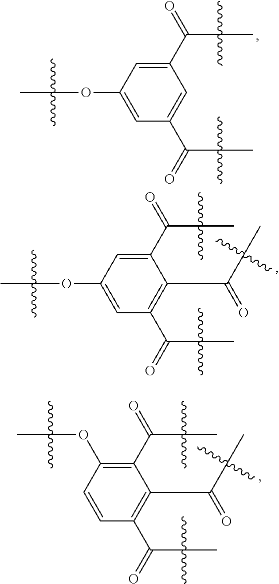
and a combination thereof, wherein n1, n2, n3, n4, and n5 are 2.

10. The bPACE polymer of claim 8, wherein B′, independently for each occurrence, comprises a structure selected from: 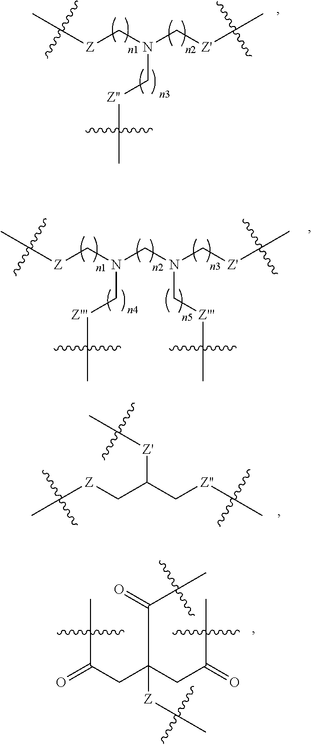
and a combination thereof.

11. The bPACE polymer of claim 10, wherein B′, independently for each occurrence, comprises a structure selected from: 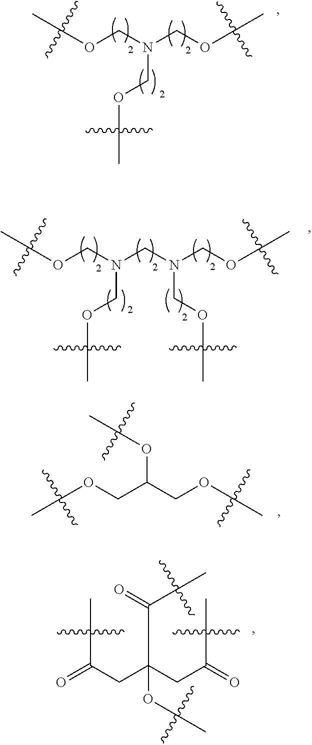
and a combination thereof.

12. The bPACE polymer of claim 1, wherein Je1, Je2, Je3, and Je4 are independently absent, —O—, —S—, —NH—, NRg, —NHC(O)—, —C(O)NH—, —OC(O)—, —C(O)O—, —NRgC(O)—, or —C(O)NRg—, wherein Rg is, independently for each occurrence, an alkyl, cycloalkyl, heterocycloalkyl, alkylaryl, alkenyl, alkynyl, aryl, or heteroaryl group.

13. The bPACE polymer of claim 1, wherein Je1, Je2, Je3, and Je4 are independently absent, —O—, —S—, —NH—, —NHC(O)—, —C(O)NH—, —OC(O)—, or —C(O)O—.

14. The bPACE polymer of claim 1, wherein Re1, Re2, Re3, and Re4 are independently absent, or substituted alkyl containing a hydroxyl group, a primary amine group, a secondary amine group, a tertiary amine group, or combinations thereof.

15. The bPACE polymer of claim 1, wherein Re1, Re2, Re3, Re4, or a combination thereof contain:
(i) a primary amine group,
(ii) a primary amine group and one or more secondary or tertiary amine groups,
(iii) a hydroxyl group, or
(iv) a hydroxyl group and one or more amine groups.

16. The bPACE polymer of claim 1, wherein:
Re1, Re2, Re3, Re4, or a combination thereof are -unsubstituted C₁-C₁₀ alkylene-Aq-unsubstituted C₁-C₁₀ alkylene-Bq, -unsubstituted C₁-C₁₀ alkylene-Aq-substituted C₁-C₁₀ alkylene-Bq, -substituted C₁-C₁₀ alkylene-Aq-unsubstituted C₁-C₁₀ alkylene-Bq, or -substituted C₁-C₁₀ alkylene-Aq-substituted C₁-C₁₀ alkylene-Bq,
wherein Aq is absent or —NR_{5b}—, and Bq is hydroxyl, primary amine, secondary amine, or tertiary amine, wherein R_{5b} is hydrogen, substituted alkyl, unsubstituted alkyl, substituted aryl, or unsubstituted aryl.

17. The bPACE polymer of claim 1, wherein Re1, Re2, Re3, Re4, or a combination thereof are selected from

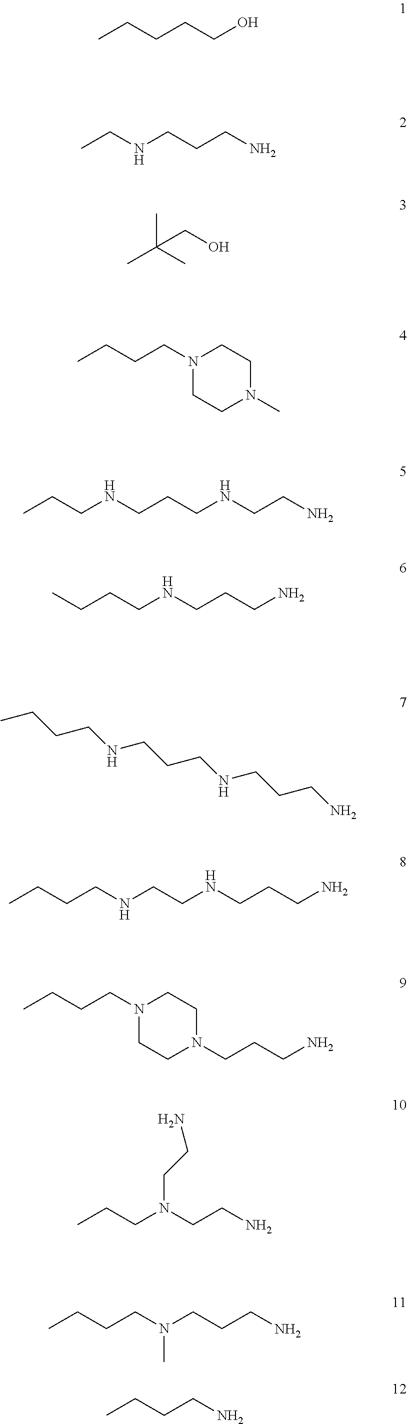

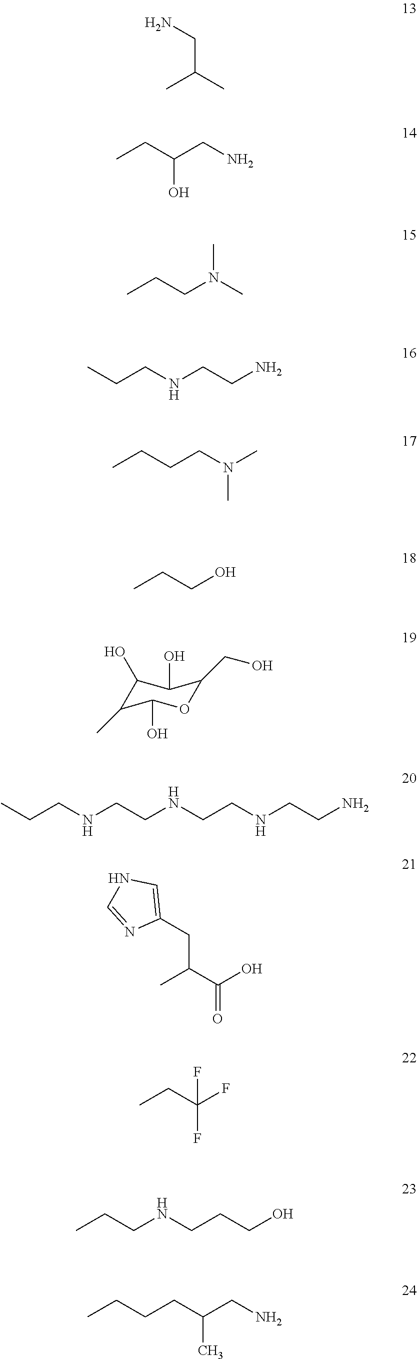

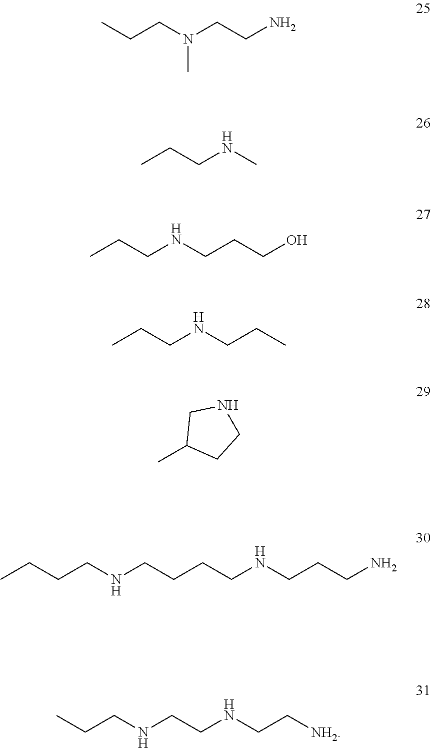

18. The bPACE polymer of claim 1, wherein Re1, Re2, Re3, Re4, or a combination thereof contains

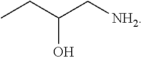

19. Particle comprising the bPACE of claim 1 and one or more therapeutic, prophylactic, or diagnostic agents.

20. The particles of claim 19, wherein:
(i) the particles are nanoparticles, microparticles, or a combination thereof, or
(ii) the one or more therapeutic, prophylactic, or diagnostic agents comprise a nucleic acid.

21. (canceled)

22. The particles of claim 20, wherein the weight ratio of the bPACE polymer to nucleic acid is from 100:1 to 5:1.

23. A pharmaceutical composition comprising the particles of claim 19 and a pharmaceutically acceptable carrier.

24. A method of administering one or more therapeutic, prophylactic, or diagnostic agents to a subject in need thereof, the method comprising administering the particles of claim 19 to the subject.

25. A method of administering one or more therapeutic, prophylactic, or diagnostic agents to a subject in need thereof, the method comprising administering the composition of claim 23 to the subject.
